(19) United States
(12) Patent Application Publication
(10) Pub. No.: US 2026/0308626 A1
(43) Pub. Date: Oct. 8, 2026

(54) ENGINEERED BACTERIOPHAGE PARTICLE AND METHOD OF GENE THERAPY BY USING SAME

(71) Applicant: ACADEMIA SINICA, Taipei City (TW)

(72) Inventors: Yun MOU, Taipei City (TW); Chia-Yi KAO, Taipei City (TW)

(73) Assignee: ACADEMIA SINICA, Taipei City (TW)

(21) Appl. No.: 19/485,468

(22) PCT Filed: May 20, 2024
(86) PCT No.: PCT/US2024/030215

§ 371 Date: Nov. 18, 2025

Related U.S. Application Data
(60) Provisional application No. 63/467,831, filed on May 19, 2023.

(51) Int. Cl. C12N 15/86; A61P 35/00

(52) U.S. Cl. CPC C12N 15/86; A61P 35/00; C12N 2795/14122; C12N 2795/14143

(57) ABSTRACT

Provided is an engineered bacteriophage particle for gene delivery including an engineered phagemid having a restructure f1 origin of replication, an M13 phage capsid encapsulating the engineered phagemid, and a cell-targeting ligand displayed on the M13 phage capsid. Also provided is a vector system for producing the engineered bacteriophage particle and a method of gene therapy by using the engineered bacteriophage particle.

### CROSS-REFERENCE TO RELATED APPLICATION

This application claims priority to U.S. provisional patent application No. 63/467,831, filed on May 19, 2023, the entire content of which is incorporated herein by reference.

### TECHNICAL FIELD

The present disclosure relates to engineered bacteriophage particles for gene delivery, in particular, engineered bacteriophage particles derived from M13 phage.

### BACKGROUND

Gene therapy based on viral vectors has emerged as a promising modality for treating various human diseases from Mendelian disorders to cancers. In recent years, several gene therapies have been approved for clinical uses, including Luxtuma for treating Leber's congenital amaurosis (LCA) and Zolgensma for treating spinal muscular atrophy (SMA)[¹⁻³]. These commercial products as well as other gene-therapy agents in clinical trials primarily utilize three types of human viral vectors: adenovirus, adeno-associated virus (AAV), and retrovirus/lentivirus. These human viruses share the advantages of high transduction efficiency to many human cell types, but they also face several critical limitations, such as (1) low infection rates to some refractory cell types, (2) poor cell-type specificity (i.e., the off-target problem), (3) existence of neutralization antibodies in patients, and (4) finite transgene capacity.

The first problem can be somewhat ameliorated by capsid engineering. For example, the AAV capsids have been evolved to improve the transduction efficiency toward human hepatocytes, neurons, and muscle^{[4-6]}. However, such engineering works often rely on creating large libraries coupled with high-throughput selection methods, which is labor-intensive, time-consuming, and technique-demanding. In addition to the aforementioned difficulties, the large-scale production of human viral vectors is also very challenging and expensive, resulting in extremely high costs and low capacity of current gene therapies.

The M13 phage has been proposed as an alternative vector for gene therapy because of the great engineerabilty, the economic large production, and the absence of existing neutralization antibodies in human bodies. The M13 particle has 3 to 5 copies of pIII minor capsid proteins, which are highly amenable for displaying a variety of exogenous proteins, including antibodies. However, the major hurdle of the M13 vector for gene therapy is still the insufficient transduction efficiency as compared to the human viral vectors. In general, the M13 vector can only transduce human cells with less than 10% efficiency^{[7]}.

Hence, there is an unmet need in the art to develop viral vectors that have desired transduction efficiency for human cells and can be useful for safe and site-specific gene integration for applications such as gene therapy.

### SUMMARY

In view of the foregoing, the present disclosure provides a viral vector for gene delivery which exhibits excellent infection specificity and optimal transduction efficiency toward eukaryotic cells and is thus useful for gene therapy.

In at least one embodiment of the present disclosure, an engineered phagemid for gene delivery is provided. The engineered phagemid comprises an f1 origin of replication and a transgene expression cassette. In some embodiments, the engineered phagemid of the present disclosure has a length less than about 8,000 nucleotides (nt) and is free from encoding an M13 phage structural protein required for M13 phage assembly.

In at least one embodiment of the present disclosure, the engineered phagemid has a length ranging from about 1,000 nt to about 6,000 nt. In some embodiments, the length of the engineered phagemid may be at least 1,000 nt, at least 1,500 nt, at least 2,000 nt, at least 2,500 nt, at least 3,000 nt, at least 3,500 nt, at least 4,000 nt, at least 4,500 nt, or at least 5,000 nt. In some embodiments, the length of the engineered phagemid may be less than 6,500 nt, less than 6,000 nt, less than 5,500 nt, less than 5,000 nt, less than 4,500 nt, less than 4,000 nt, less than 3,500 nt, less than 3,000 nt, less than 2,500 nt, or less than 2,000 nt. In some embodiments, the length of the engineered phagemid may range from about 1,000 nt to about 5,000 nt, about 1,000 nt to about 4,000 nt, about 1,000 nt to about 3,000 nt, about 2,000 nt to about 5,000 nt, or about 2,000 nt to about 4,000 nt.

In at least one embodiment of the present disclosure, the f1 origin of replication may be a naturally occurring f1 origin of replication, which has a phage packaging signal, loop B-C, domain A, and domain B. In some embodiments, the f1 origin of replication may comprise the phage packaging signal, the loop B-C, the domain A, and the domain B in order of 5′ to 3′.

In at least one embodiment of the present disclosure, the f1 origin of replication may be genetically modified to further comprise a truncated domain A. In some embodiments, the f1 origin of replication may be genetically modified to lack at least one of loop B-C and domain B.

In at least one embodiment of the present disclosure, the transgene expression cassette is configured to express the transgene in a eukaryotic cell. In some embodiments, the transgene expression cassette may be integrated into the f1 origin of replication.

In at least one embodiment of the present disclosure, the f1 origin of replication may be a restructured f1 origin of replication comprising the domain A, the domain B, the transgene expression cassette, the phage packaging signal, the loop B-C, and the truncated domain A in order of 5′ to 3′.

In at least one embodiment of the present disclosure, the f1 origin of replication may be a restructured f1 origin of replication comprising the domain A, the domain B, the transgene expression cassette, the phage packaging signal, and the truncated domain A in order of 5′ to 3′.

In at least one embodiment of the present disclosure, the f1 origin of replication may be a restructured f1 origin of replication comprising the domain A, the transgene expression cassette, the phage packaging signal, and the truncated domain A.

In at least one embodiment of the present disclosure, the engineered phagemid comprises the transgene expression cassette and a restructured f1 origin of replication having at least 70% (e.g., at least 80%, at least 90%, at least 95%, and at least 99%) sequence identity to SEQ ID NO. 1, wherein the transgene expression cassette is integrated into the restructured f1 origin of replication. In some embodiments, the transgene expression cassette and a restructured f1 origin of replication are represented by SEQ ID NO. 1, wherein the transgene expression cassette is integrated into the nucleotide sequence of SEQ ID NO. 1.

In at least one embodiment of the present disclosure, the engineered phagemid comprises the transgene expression cassette and at least one nucleotide sequence having at least 70% (e.g., at least 80%, at least 90%, at least 95%, and at least 99%) sequence identity to one of SEQ ID NOs. 2 to 6, wherein the at least one nucleotide sequence serves as the signal for initiation of replication of the engineered phagemid that has the same function as the corresponding nucleotide sequence of SEQ ID NOs. 2 to 6. In some embodiments, the engineered phagemid comprises the transgene expression cassette and at least one nucleotide sequence represented by one or more of SEQ ID NOs. 2 to 6.

In at least one embodiment of the present disclosure, the engineered phagemid comprises the transgene expression cassette and a restructured f1 origin of replication comprising a first nucleotide sequence having at least 70% (e.g., at least 80%, at least 90%, at least 95%, and at least 99%) sequence identity to SEQ ID NO. 10 and a second nucleotide sequence having at least 70% (e.g., at least 80%, at least 90%, at least 95%, and at least 99%) sequence identity to SEQ ID NO. 11, wherein the first and second nucleotide sequences serve as the signal for initiation of replication of the engineered phagemid that have the same functions as SEQ ID NOs. 10 and 11. In some embodiments, the restructured f1 origin of replication comprises a first nucleotide sequence represented by SEQ ID NO. 10 and a second nucleotide sequence represented by SEQ ID NO. 11. In some embodiments, the engineered phagemid comprises the first nucleotide sequence, the transgene expression cassette, and the second nucleotide sequence in order of 5′ to 3′.

In at least one embodiment of the present disclosure, the transgene expression cassette comprises a functional element required for expression of the transgene in a eukaryotic cell. In some embodiments, the functional element is at least one of a promoter and a poly-A sequence. In some embodiments, the promoter is a mammalian expression promoter. In some embodiments, the promoter is selected from the group consisting of a cytomegalovirus (CMV) major immediate-early promoter, a simian virus 40 (SV40) promoter, a β-actin promoter, an albumin promoter, an elongation factor 1-α (Ef1-α) promoter, a PγK promoter, an MFG promoter, and a Rous sarcoma virus promoter.

In at least one embodiment of the present disclosure, the transgene expression cassette may be codon optimized for expression in a eukaryotic cell. In some embodiments, the eukaryotic cell may be a mammalian cell.

In at least one embodiment of the present disclosure, the engineered phagemid may further comprise a selectable marker and/or an enhancer. In some embodiments, the selectable marker may be an antibiotic resistance gene, such as an ampicillin resistance gene, a kanamycin resistance gene, a gentamycin resistance gene, a chloramphenicol resistance gene, a spectinomycin resistance gene, a streptomycin resistance gene, a tetracycline resistance gene, a blasticidin S resistance gene, a hygromycin resistance gene, a puromycin resistance gene, and a Zeocin resistance gene.

In at least one embodiment of the present disclosure, a vector system of producing an engineered bacteriophage particle for gene delivery is also provided. The vector system comprises the above engineered phagemid and a helper plasmid comprising an M13 phage genome, a prokaryotic origin of replication, and a nucleic acid encoding a cell-targeting ligand. In some embodiments, the helper plasmid encodes an M13 phage structural protein required for packaging the engineered phagemid in a prokaryotic cell, thereby forming the engineered bacteriophage particle for gene delivery. In some embodiments, the prokaryotic cell may be Escherichia coli. (E. coli).

In at least one embodiment of the present disclosure, the M13 phage genome comprises genes of pI, pII, pIII, pIV, pV, pVI, pVII, pVIII, pIX, and pX. In some embodiments, the nucleic acid encoding the cell-targeting ligand is fused into the pIII gene.

In at least one embodiment of the present disclosure, the prokaryotic origin of replication is selected from the group consisting of p15a, ColE1, pSC101, pMB1, R6K and RK2.

In at least one embodiment of the present disclosure, the helper plasmid comprises a disrupted f1 origin of replication or is free of an f1 origin of replication.

In at least one embodiment of the present disclosure, the helper plasmid further comprises a selectable marker, such as a kanamycin resistance gene.

In at least one embodiment of the present disclosure, an engineered bacteriophage particle for gene delivery is also provided. The engineered bacteriophage particle comprises the above engineered phagemid, an M13 phage capsid encapsulating the engineered phagemid, and a cell-targeting ligand displayed on the M13 phage capsid.

In at least one embodiment of the present disclosure, the engineered bacteriophage particle has a length ranging from about 300 nm to about 600 nm. In some embodiments, the length of the engineered bacteriophage particle may be about 300 nm, about 400 nm, about 500 nm, or about 600 nm.

In at least one embodiment of the present disclosure, the M13 phage capsid comprises proteins of pIII, pVI, pVIII, pIX, and pVII. In some embodiments, the cell-targeting ligand may be displayed on the pIII protein of the M13 phage capsid.

In at least one embodiment of the present disclosure, the cell-targeting ligand is selected from the group consisting of a peptide, an antibody, an antibody fragment, and a non-proteinaceous molecule. In some embodiments, the cell-targeting ligand may be a tumor-targeting ligand, such as an EGFR ligand and a vascular endothelial growth factor receptor (VEGFR) ligand. In some embodiments, the EGFR ligand may be selected from the group consisting of EGF, HB-EGF, TGFα, and betacellulin.

In at least one embodiment of the present disclosure, the engineered bacteriophage particle is produced by the above vector system in a prokaryotic cell.

In at least one embodiment of the present disclosure, a method of delivering a transgene to a eukaryotic cell is also provided. The method comprises contacting the eukaryotic cell with the above engineered bacteriophage particle. In some embodiments, the eukaryotic cell is a PrimPol-overexpressing and/or DMBT1 knockout cell.

In at least one embodiment of the present disclosure, the transgene carried by the engineered bacteriophage particle encodes a therapeutic protein in the eukaryotic cell transduced with the engineered bacteriophage particle. In some embodiments, the therapeutic protein may be GSDMD or a fragment thereof. In some embodiments, the therapeutic protein may be a fragment crystallizable region (Fc region) inducing antibody-dependent cell-mediated cytotoxicity or antibody-dependent cellular phagocytosis in the eukaryotic cell transduced with the engineered bacteriophage particle. In some embodiments, the therapeutic protein may be an IgG Fc fragment.

In at least one embodiment of the present disclosure, the transgene carried by the engineered phagemid is a gene-editing molecule exerting gene editing in the eukaryotic cell transduced with the engineered bacteriophage particle.

In at least one embodiment of the present disclosure, a method of preventing or treating a disease susceptible to amelioration by gene therapy is also provided. The method comprises administering a therapeutically effective amount of the above engineered bacteriophage particle to a subject in need thereof.

In at least one embodiment of the present disclosure, the disease susceptible to amelioration by gene therapy is a cancer or a genetic disorder. In some embodiments, the gene therapy may be immunogen therapy.

In at least one embodiment of the present disclosure, the cancer includes, but is not limited to, skin cancer, breast cancer, head and neck cancer, lung cancer, stomach cancer, pancreatic cancer, ovarian cancer, cervical cancer, uterine cancer, kidney cancer, bladder cancer, colon cancer, prostate cancer, central nervous system cancer, ocular melanoma, neuroblastoma, multiple myeloma, and lymphoma.

In at least one embodiment of the present disclosure, the genetic disorder includes, but is not limited to, Duchenne muscular dystrophy, limb-girdle muscular dystrophy, spinal muscular atrophy, myotubular myopathy, Pompe disease, glycogen storage disease, Leber's congenital amaurosis, amyotrophic lateral sclerosis, color blindness, type 1 diabetes, and Fabry disease.

In the present disclosure, the engineered bacteriophage particle provided herein is effective in delivering a gene to a target cell, e.g., a eukaryotic cell. The engineered bacteriophage particle has an unprecedented transduction efficiency toward human cells, which is comparable or even superior to the AAV vectors. Also, the engineered bacteriophage particle exhibits superior specificity compared to the AAV vectors. Hence, the engineered bacteriophage particle can be useful for specifically treating disease cells without the impact on the healthy cells, thereby effectively preventing or treating a disease susceptible to amelioration by gene therapy.

### BRIEF DESCRIPTION OF THE DRAWINGS

For a full understanding of this disclosure, reference should be made to the following detailed descriptions, taken in connection with the accompanying drawings.

FIGS. 1A to 1N illustrate that the transduction efficiency of ligand-displayed M13 phage exhibits binary specificity depending on the receptor expression level in cells. FIG. 1A is a diagram showing multivalent display of EGF on pIII of M13 bacteriophage, in which all copies of pIII are fused with the human EGF gene in the helper plasmid. FIG. 1B shows the various levels of EGFR expression in five cell lines (i.e., HeLa, PC3, Caco-2, HepG2, and HEK293T). FIGS. 1C to 1F show the transduction efficiency of EGF-phage^{6k} (GFP) (FIGS. 1C and 1E) and TGFα-phage^{6k}(GFP) (FIGS. 1D and 1F) against the five cell lines determined by the GFP reporter expression, respectively, in which the GFP expression is analyzed by flow cytometry after 5 days of transduction. FIG. 1G shows the EGFR binding strength of six EGFR ligands (i.e., EGF, HB-EGF, TGFα, betacellulin, amphiregulin, and epiregulin) displayed on phages, in which each phage is added to the EGFR-coated plate and measured for the binding using ELISA. FIGS. 1H to 1J show the transduction efficiency of the M13 phage displaying one of the six EGFR ligands in HeLa cells, in which the gene delivery efficiency is analyzed by flow cytometry 5-day post-transduction. FIGS. 1K and 1L show the immunostaining of EGF-phage and EGFR in the HeLa cells, in which FIG. 1K shows the representative confocal images of HeLa cells after 2 hours of the EGF-phage challenge (red: antibody staining of the M13 phage; green: antibody staining of EGFR; blue: DAPI staining), and FIG. 1L shows the cross-section analysis of the EGF-phage^{6k} and the EGFR signals from the arrow in FIG. 1K. FIG. 1M shows the validation of the EGFR-overexpressing HEK293T cell lines by flow cytometry, in which the cell surface EGFR expression of EGFR-HEK293T and EGFR(NLSm)-HEK293T is immunostained and quantified by flow cytometry. FIG. 1N shows the transduction efficiency of EGF-phage^{6k} (GFP) against the EGFR-HEK293T, the EGFR(NLSm)-HEK293T, and the parental HEK293T cells.

FIGS. 2A to 2H illustrate that the phage transduction efficiency is enhanced by the genotoxic treatment and the PrimPol up-regulation. FIG. 2A shows the transduction efficiency of EGF-phage^{6k} (GFP) against the HeLa and PC3 cells with and without the camptothecin (CPT) treatment. FIG. 2B shows the cell cycle analysis of HeLa cells with and without the camptothecin (CPT) treatment. FIG. 2C shows the PrimPol transcript level of HeLa cells with and without the camptothecin (CPT) treatment. FIG. 2D shows the validation of the PrimPol-overexpressing cell lines by quantitative PCRs, in which the PrimPol-overexpressing cell lines of HeLa, PC3, and EGFR-HEK293T are created by lentiviral transduction and antibiotic selection, and the PrimPol transcript level is quantified by qPCR and normalized to the parental cell line. FIGS. 2E to 2G show the transduction efficiency of EGF-phage against HeLa (FIG. 2E), HEK293T (FIG. 2F), and PC3 cells (FIG. 2G) with and without the PrimPol overexpression, in which the EGF-phage^{6k} (GFP) is used for the HeLa experiments, the EGF-TransPhage (GFP) is used for the HEK293T experiments, and TGFα-TransPhage (GFP) is used for the PC3 experiments. FIG. 2H shows the confocal images of the GFP gene delivery by EGF-phage, in which the transduction experiments are performed on HeLa cells or PrimPol-HeLa cells.

FIGS. 3A to 3L illustrate that the down-regulation of DMBT1 enhances the phage transduction as revealed by the RNA-seq data. FIG. 3A shows the PrimPol-HeLa^{hi} cells sorted from the double-positive high-expression population (red area), in which the EGF-phage carrying GFP or mCherry is singly or sequentially applied to the PrimPol-HeLa cells. FIG. 3B shows the transduction efficiency of EGF-phage (GFP) in HeLa, PrimPol-HeLa, and PrimPol-HeLa^{hi} cells.

FIGS. 3C and 3D show the comparison of the expression levels of PrimPol and EGFR in PrimPol-HeLa and PrimPol-HeLa^{hi} determined by qPCR and flow cytometry, respectively. FIG. 3E shows the volcano plot of the transcriptome comparison between PrimPol-HeLa and PrimPol-HeLa^{hi}. FIG. 3F shows the MA plot of the transcriptome comparison between PrimPol-HeLa and PrimPol-HeLa^{hi} (red: up-regulated transcripts (fold change >2) with P values <0.05; blue: down-regulated transcripts (fold change <0.5) with P values <0.05). FIG. 3G shows the representative biological process gene ontology (GO) terms of the RNA-seq data. FIG. 3H shows the top 5 down-regulated genes in each representative GO term. FIG. 3I shows the genes that the Ingenuity Pathway Analysis (IPA) predicted to enhance the viral infection in the PrimPol-HeLa^{hi} cells and their fold changes. FIG. 3J shows the ELISA assay results of phage binding to the conditioned medium from HeLa and DMBT1-KO HeLa. FIG. 3K shows the EGF-phage transduction efficiency in HeLa and DMBT1-KO HeLa. FIG. 3L shows the comparison of the phage transduction efficiencies in the parental HeLa cells (left panel) and the DMBT1-KO HeLa cells (right panel).

FIGS. 4A to 4D illustrate the inverse correlation between the phage length and the transduction efficiency. FIG. 4A shows the electron microscope images for the EGF-phage with different lengths. FIG. 4B shows the linear relationship between the phage length and the encapsulated genome size. FIGS. 4C and 4D show the transduction efficiency of EGF-phage^{4k}, EGF-phage^{6k}, and EGF-phage^{8k} in the PrimPol-HeLa cells, in which the phage is prepared by the M13KO7 helper (FIG. 4C) or the f1-less helper plasmid (FIG. 4D).

FIGS. 5A to 5H illustrate that the minimization of phage length by restructuring the f1 origin domains leads to the optimum transduction efficiency. FIG. 5A shows the schematic maps of the ssDNAs encapsulated in phage^{2k}, phage^{4k}, phage^{2k+domainB}, and phage^{2k+domainB+loopBc} (TransPhage), respectively (PS: packaging signal; tDomain A: truncated domain A). FIG. 5B shows the comparison of the transduction efficiencies between EGF-phage^{2k} and EGF-phage^{4k}, in which the phage is prepared by the M13KO7 helper or the f1-less helper plasmid. The transduction efficiencies of EGF-phage^{2k} and EGF-phage^{4k} to the PrimPol-HeLa cells are determined by flow cytometry, and an equal production unit of phage (total phage production from 3 mL bacteria culture) is applied to the cells. The GFP expression is measured after 5 days of the phage transduction. FIG. 5C shows the comparison of EGF-phage^{2k} and EGF-phage^{2k+domainB} in transducing the PrimPol-HeLa cells, in which the phage is prepared by the M13KO7 helper or the f1-less helper plasmid. FIG. 5D shows the comparison of EGF-phage^{2k+domainB} and EGF-phage^{2k+domainB+loopBC} in transducing the PrimPol-HeLa^{hi} cells. FIG. 5E shows the schematic map of TransPhage and the explicit DNA sequence thereof (i.e., SEQ ID NO. 1), in which the triangles mark the points of cleavages and circulation (blue: domain A (initiator); cyan: domain B (positive-strand enhancer); green: gene of interest, including a promoter, the open reading frame, and a poly A signal; orange: packaging signal; yellow: loop B-C (negative-strand initiator); purple: truncated domain A (terminator)). FIGS. 5F and 5G show the comparison of EGF-TransPhage, EGF-phage^{4k}, EGF-phage^{6k}, and EGF-phage^{8k} in transducing the PrimPol-HeLa^{hi} cells, in which an equal MOI (10⁷ phage particle/cell) (FIG. 5F) or an equal phage weight (0.13 ng/cell) (FIG. 5G) is used for each experiment, and the P values between all groups are <0.001. FIG. 5H shows the transduction efficiency of TransPhage against the parental HeLa cells, in which the EGF-TransPhage that carried mCherry or membrane-bound human Fc is used to transduce the parental HeLa cells, and the GFP or the membrane-bound Fc level is determined by flow cytometry after 5 days (red: with transduction; blue: without transduction).

FIGS. 6A to 6J illustrate the comparison of the transduction efficiency and specificity of AAV vectors and TransPhage. FIG. 6A shows the relationship between the transduction efficiency and the MOI of EGF-TransPhage (GFP), in which the EGF-TransPhage (GFP) at various MOIs are applied to the PrimPol-HeLa^{hi} cells. FIGS. 6B to 6D show the transduction efficiencies of AAV6, AAV8, AAV9, and EGF-TransPhage in PrimPol-HeLa^{hi} at MOI of 10⁷, in which the GFP expression profiles are measured by flow cytometry (FIG. 6B), and the transduction efficiency is presented by the GFP+ population (FIG. 6C) and the mean fluorescence intensity (FIG. 6D). FIGS. 6E to 6G show the same as FIGS. 6B to 6D but a MOI of 10⁶ is used. FIGS. 6H to 6J show the transduction efficiencies of AAV6, AAV8, AAV9, and EGF-TransPhage in HEK293T cells at MOI of 10⁷.

FIGS. 7A to 7C illustrate the in vitro applications of TransPhage for cancer therapy. FIG. 7A shows that the N-terminal domain of GSDMD is delivered by EGF-phage^{6k} or TransPhage into the PrimPol-HeLa^{hi} cells, and the cell viability is measured five days later. FIG. 7B shows that the membrane-bound human Fc is delivered by TransPhage and expressed on the PrimPol-HeLa^{hi} cells as measured by flow cytometry (red: EGF-TransPhage transduction; blue: no transduction). FIG. 7C shows that the NK92 cells with or without CD16 expression are co-cultured with the HeLa cells with or without the membrane-bound human Fc expression, and the cytotoxicity of the target cells is measured at various effector-to-target cell (E:T) ratios.

FIGS. 8A and 8B illustrate the transduction efficiency of EGF-TransPhage in xenograft tumors, in which the EGF-TransPhage that carries an mCherry gene is intratumorally administrated into subcutaneous PC3 tumors in nude mice, and the tumors are homogenized and analyzed for the mCherry expression in the CD45+ and CD45− cell populations. A blank EGF-TransPhage without an mCherry gene is used as a negative control.

FIGS. 9A to 9C illustrate that TransPhage carrying the membrane-bound Fc triggers tumor necrosis and inhibits the tumor growth. The nude mice are inoculated with the human prostate cancer cell line PrimPol-PC3 subcutaneously and treated intratumorally using EGF-TransPhage that carries mCherry, membrane-bound human Fc (IgG1), or membrane-bound mouse Fc (IgG2a) every other day for four times (Day 0, 2, 4, and 6). FIG. 9A shows the representative pictures of individual treatments on day 2 and day 10. FIG. 9B shows the tumor growth curves. FIG. 9C shows the mouse survival rates, in which tumor size >1,000 mm³ is considered death. Day 0 is defined as the day of the first TransPhage treatment when the tumor size was about 100 mm³. A total of four treatments are administered on days 0, 2, 4, and 6.

### DETAILED DESCRIPTION

The description discloses some embodiments in such a detail that a person skilled in the art is able to utilize the embodiments based on the disclosure. Not all steps or features of the embodiments are discussed in detail, as many of the steps or features will be obvious for a person skilled in the art based on this disclosure.

As used in this disclosure, the singular forms “a,” “an,” and “the” include plural referents unless the content clearly dictates otherwise. As used herein, the term “and” is intended to be inclusive unless otherwise indicated. As used herein, the term “or” is generally employed in its sense including “and/or” unless the context clearly dictates otherwise.

As used herein, the term “about” refers to a degree of deviation for a property, composition, amount, value, or parameter as identified, such as deviations based on experimental errors, measurement errors, approximation errors, calculation errors, standard deviations from a mean value, routine minor adjustments, and so forth.

As used herein, the terms “comprising,” “having,” “including,” and “containing” are to be construed as open-ended terms (i.e., meaning “including, but not limited to”) unless otherwise noted.

The present disclosure provides an engineered bacteriophage particle for gene delivery, which is an M13 phage-derived bacteriophage particle. M13 phage is a filamentous bacteriophage composed of circular single stranded DNA (ssDNA) which is 6,407 nucleotides long encapsulated in approximately 2,700 copies of the major coat protein pVIII and capped with 3 to 5 copies of four different minor coat proteins (pIX, pVII, pVI, and pIII) on the ends. The minor coat protein pIII attaches to the receptor at the tip of the F pilus of the host Escherichia coli.

The present disclosure also provides a method of preventing or treating a disease susceptible to amelioration by gene therapy, comprising administering a therapeutically effective amount of the engineered bacteriophage particle to a subject in need thereof.

As used herein, the term “gene therapy” means a treatment based on the genetic modification of cells to produce a therapeutic effect by the delivery of nucleic acids into patient's cells. In some cases, the whole or part of a gene is defective or missing from birth, or a gene can change or mutate during life. Any of these variations can disrupt how proteins are synthesized, which can contribute to health problems or diseases. By gene therapy, a defective gene or genetic sequence that causes a medical problem can be replaced with a healthy version; genes (or sequences) can also be added to help the body fight or treat diseases; or genes (or sequences) that are causing problems can be knocked down or knocked out. Thereby, gene therapy can be used to treat inherited or acquired diseases.

As used herein, the terms “patient,” “individual,” “host,” and “subject” are used interchangeably. The term “subject” means a human or an animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In some embodiments, the subject is a mammal, e.g., a primate such as a human.

As used herein, the term “treat,” “treating,” or “treatment” encompasses partially or completely preventing, ameliorating, mitigating, and/or managing a symptom, a disorder, or a condition associated with a disease. The term “treat,” “treating,” or “treatment” as used herein refers to application or administration of one or more therapeutic agent or surgery to a subject, who has a symptom, a disorder, or a condition associated with a disease, with the purpose to partially or completely alleviate, ameliorate, relieve, delay onset of, inhibit progression of, reduce severity of, and/or reduce incidence of one or more symptoms, disorders, or conditions associated with the disease. Treatment may be administered to a subject who exhibits only an early sign of such symptoms, disorders, and/or conditions for the purpose of decreasing the risk of developing the symptoms, disorders, and/or conditions associated with a disease.

As used herein, the term “preventing” or “prevention” refers to preventive or avoidance measures for a disease or symptoms or conditions of a disease, which include, but are not limited to, applying or administering one or more active agents to a subject who has not yet been diagnosed as a patient suffering from the disease or the symptoms or conditions of the disease but may be susceptible or prone to the disease. The purpose of the preventive measures is to avoid, prevent, or postpone the occurrence of the disease or the symptoms or conditions of the disease.

As used herein, the phrase “a therapeutically effective amount” refers to the amount of an active ingredient (e.g., an engineered bacteriophage particle) that is required to confer a desired effect on the treated subject. Effective doses will vary, as recognized by one of ordinary skill in the art, depending on routes of administration, excipient usage, the possibility of co-usage with other treatment, and the condition to be treated.

As used herein, the term “sequence identity” means the percentage that the nucleotide residues of a candidate nucleic acid fragment are completely identical to the nucleotide residue of a reference nucleic acid fragment. When performing the above comparison, said candidate nucleic acid fragment may be aligned, and the gaps may be introduced as necessary, so as to form the highest sequence identity between the two sequences. It should be understood that compared to the nucleotide sequence of a reference nucleic acid fragment in the present disclosure, the nucleotide sequence of a candidate nucleic acid fragment, which is modified (e.g., deleted, substituted, or added) at least a part in the sequence, is also within the scope of the present disclosure, as long as the resulting candidate nucleic acid fragment has substantially the same biological functions as the reference nucleic acid fragment.

Many examples have been used to illustrate the present disclosure. The examples below should not be taken as a limit to the scope of the present disclosure.

### Examples

### Materials and Methods

The materials and methods used in the following Examples 1 to 8 are described in detail below. The materials used in the present disclosure but unannotated herein are commercially available.

### (1) Phagemid and Helper Constructs

For displaying proteins on pIII, the DNA fragments of different epidermal growth factor receptor (EGFR) ligands (epidermal growth factor (EGF), transforming growth factor-α (TGFα), heparin-binding EGF (HB-EGF), betacellulin, amphiregulin, and epiregulin) were fused between the pIII signal peptide (1-18 amino acids (a.a.)) and the truncated pIII gene (264-424 a.a.) in the M13KO7 helper vector or the f1-less helper. The f1-less helper was created by removing the f1 origin from M13KO7.

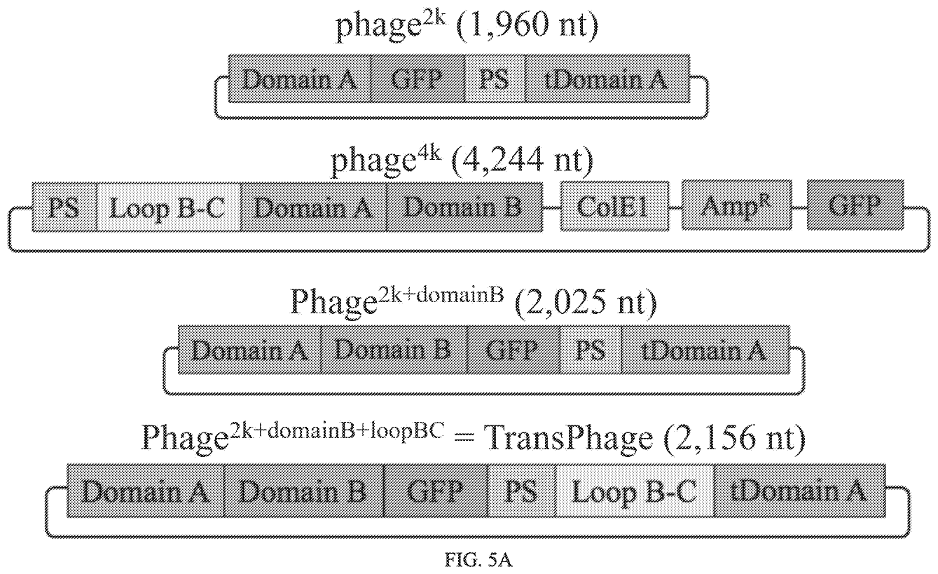

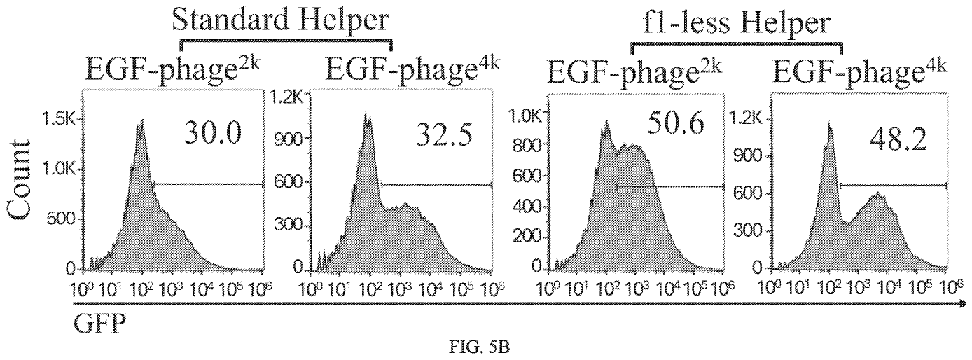

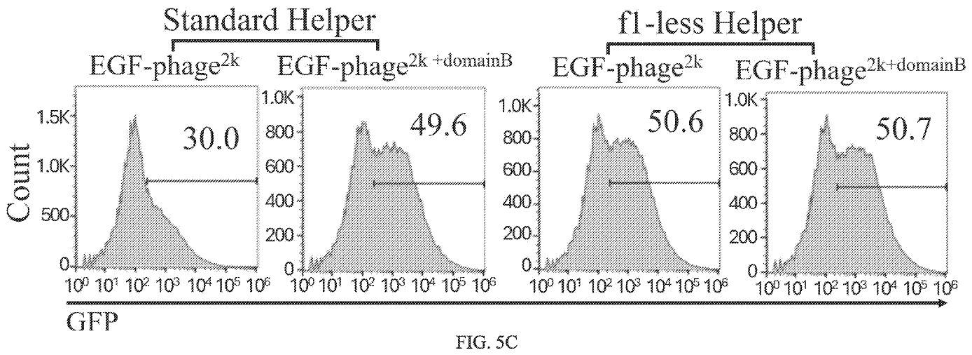

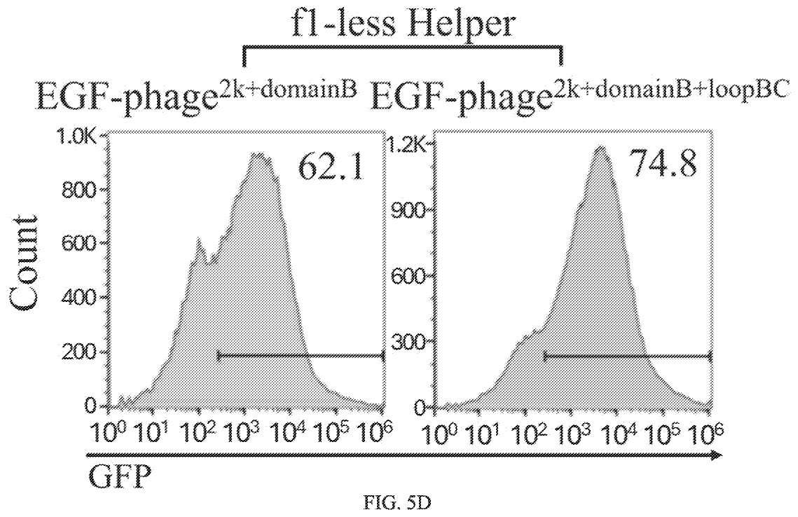

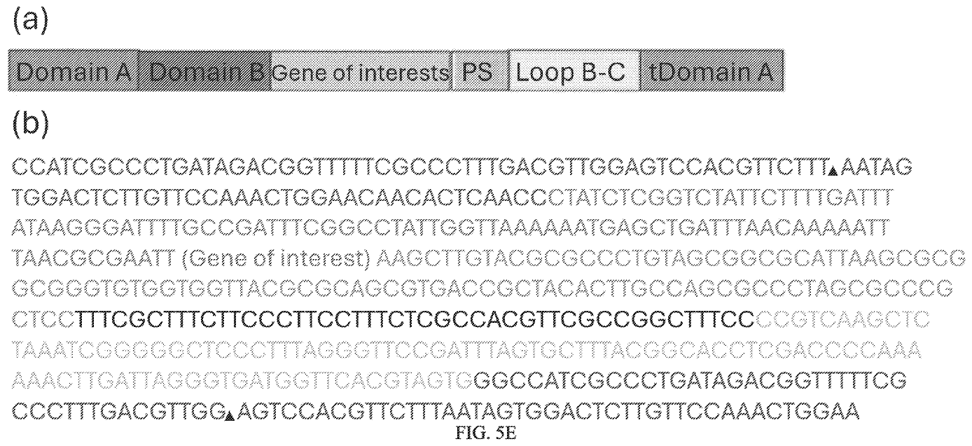

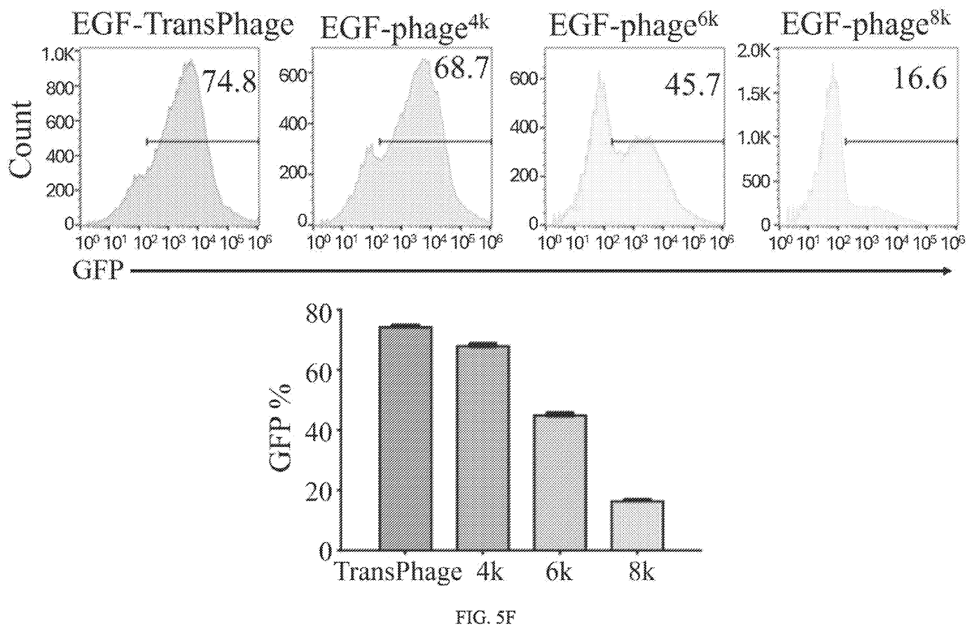

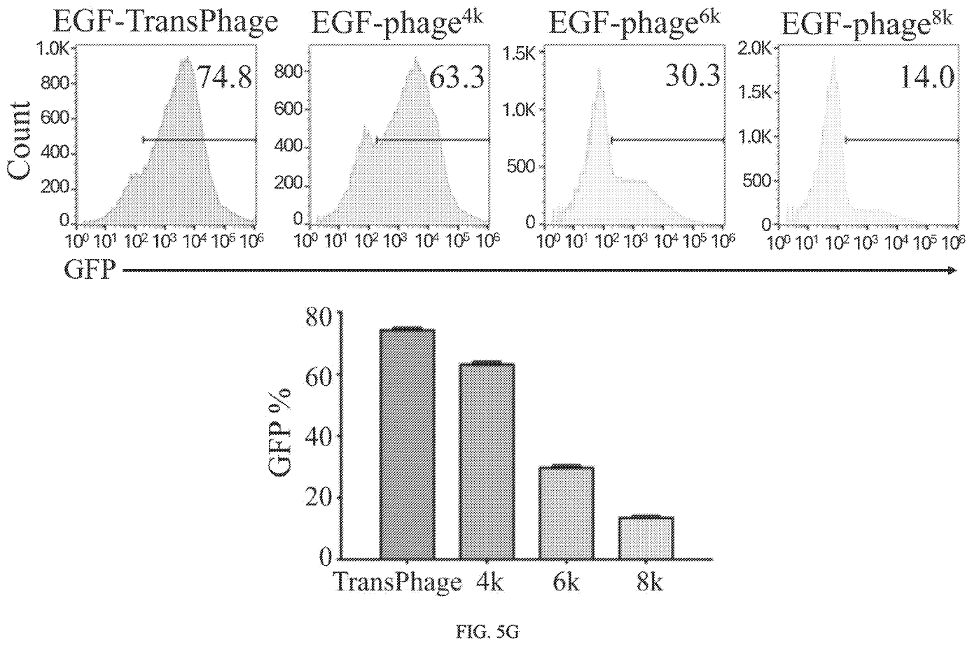

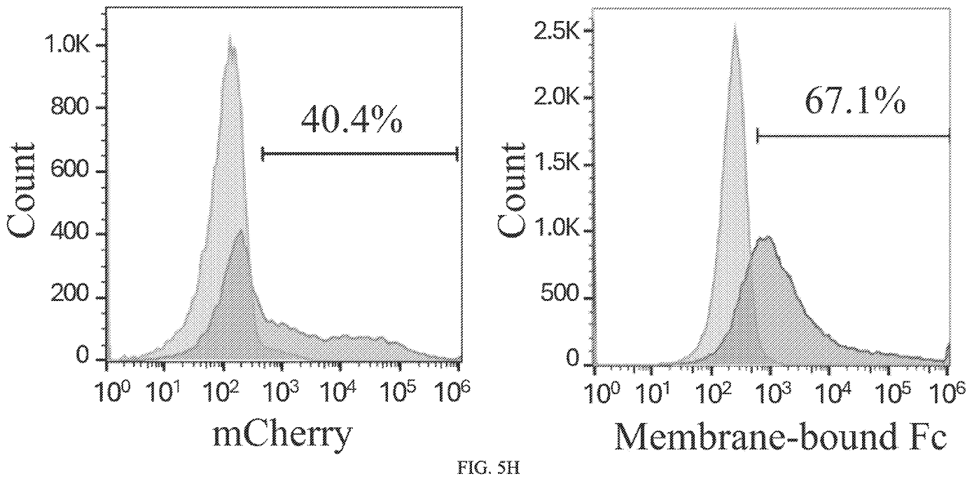

For the EGF-phage^{6k} (GFP) construct, the green fluorescent protein (GFP) gene was cloned into the pcDNA3 vector, resulting in a phagemid size of 6,133 nucleotides (nt). The EGF-phage^{4k}(GFP) or the EGF-phage^{8k} (GFP) phagemid was created by deleting the neomycin resistance marker or inserting a spacer sequence from EGF-phage^{6k} (GFP), respectively. For TransPhage, the DNA sequence represented by SEQ ID NO. 1 was inserted into the pUC19 vector with the ColE1 origin and the Amp resistance. As shown in FIG. 5E, the TransPhage comprises the domain A (SEQ ID NO. 2), the domain B (SEQ ID NO. 3), the packaging signal (PS, SEQ ID NO. 4), the loop B-C(SEQ ID NO. 5), and the tdomain A (SEQ ID NO. 6). More specifically, in addition to the gene of interest, the TransPhage comprises SEQ ID NOs. 10 and 11.

For the pyroptosis-inducing phage, the N-terminal domain of the human gasdermin D (GSDMD) gene (1-275 a.a.) as the gene of interest was integrated into the TransPhage. For the membrane-bound Fc constructs, the mouse IgG2a Fc fragment or the human IgG1 Fc fragment was fused with a transmembrane domain from the platelet derived growth factor receptor beta (PDGFRB) to serve as the gene of interest.

### (2) M13 Bacteriophage Production and Purification

The XL1-Blue E. coli was sequentially transformed with the helper plasmid (kanamycin resistance) and the phagemid (ampicillin resistance). The double-transformed bacteria were inoculated into 35 mL 2×YT medium with 50 μg/mL kanamycin and 50 g/mL carbenicillin. The bacteria were cultured for 24 hours at 37° C. with shaking at 250 rpm. Next day, the bacteria were pelleted by centrifugation at 6,000 g for 10 min at 4° C. The supernatant was mixed with the precipitation buffer (4% PEG8000 and 0.5 M NaCl for genome >6,000 nt; 10% PEG8000 and 1.25 M NaCl for genome <6,000 nt) for 1 hour at 4° C. The phage was collected by centrifugation at 9,500 g for 20 min at 4° C., and the phage pellet was re-suspended in 1.0 mL phosphate buffered saline (PBS). The phage suspension was pelleted again with the precipitation buffer and re-suspended in 1.0 mL PBS. The double-purified phage was kept at 4° C. until further use.

### (3) AAV Production and Purification

The pseudotyped AAV6, AAV8, and AAV9 vectors were produced by a triple transfection method and purified by cesium chloride sedimentation as previously described^{[8]}. Briefly, the adenovirus helper plasmid (25 μg), the pseudotyped AAV packaging plasmid (10 μg), and the rAAV vector plasmid carrying the GFP gene driven by the CMV promoter (15 g) were co-transfected into 2×10⁷ HEK293 cells. After 2 days, the cells were harvested, and the cell pellets were resuspended in 1 mL of 150 mM NaCl-50 mM Tris-HCl (pH 8.5) and subjected to three cycles of freeze-thaw and removal of cell debris. The AAV particles were purified twice using the CsCl gradient ultracentrifugation from the cell lysate. The purified AAVs were re-suspended in PBS buffer, tittered by real-time PCR, and stored at −80° C. until further use.

### (4) Cell Culture

HEK293T and HeLa cells were maintained in Dulbecco's Modified Eagle Medium (DMEM; Gibco, #12100046) supplemented with 10% fetal bovine serum (FBS; Gibco, #10437028), and 100 units/mL penicillin and 100 μg/mL streptomycin (Pen-Strep; Gibco, #15140122). Caco-2 cells were maintained in DMEM supplemented with 20% FBS and Pen-Strep. PC-3 and HepG2 cells were maintained in RPMI-1640 medium (Gibco, #31800022) supplemented with 10% FBS and Pen-Strep. The cells were incubated in a humidified incubator at 37° C. with 5% CO₂.

### (5) Stable Cell Line Generation

The EGFR-HEK293T stable cell lines were generated by lentiviral transduction followed by puromycin selection.

The EGFR-NLSm mutant was created by replacing the nuclear localization signal (NLS) (R⁶²⁴RRHIVRKR; SEQ ID NO. 7) in EGFR to AAAHIVAAA (SEQ ID NO. 8).

The PrimPol stable cell lines were generated by transducing HeLa, PC3, and EGFR-HEK293T cells with lentivirus followed by zeocin selection.

For the lentiviral production, pCMV-dR8.91 (1.35 g), pMD2-G (0.165 g), and the transfer plasmid (1.5 g) were mixed with linear polyethylenimine (PEI, MW 25,000, 3 g/mL) in Opti-MEM (Gibco) for 30 min. The mixture was added to 9×10⁵ HEK293T cells. After 6 hours, the medium was replaced, and the supernatant was harvested 72 hours post-transfection.

For the lentiviral transduction, the target cells were incubated with the lentiviral supernatant in the presence of polybrene (8 μg/mL) for 6 hours and selected with puromycin (1 μg/mL) for 1 week or zeocin (200 μg/mL for HeLa and EGFR-HEK293T, 25 g/mL for PC-3) for 2 weeks.

### (6) Enzyme-Linked Immunosorbent Assay (ELISA)

Each well was coated with 20 nM EGFR in PBS at 4° C. for overnight. The coating solution was replaced with the blocking buffer (PBS with 0.05% Tween and 0.2% BSA) for 2 hours at room temperature. After removing the blocking buffer, 50 μL phage (about 10¹³ colony-forming unit (CFU)/mL) was added to the plate for 30 min at room temperature. The plate was washed three times with the wash buffer (PBS with 0.05% Tween) and loaded with the anti-M13 bacteriophage horseradish peroxidase (HRP) conjugated antibody (Sino Biological, #11973) for 15 min at room temperature. The plate was then washed three times with the wash buffer. Subsequently, 100 μL of the HRP 3,3′,5,5′-tetramethylbenzidine (TMB) substrate (VWR TMB Microwell Peroxidase Substrate, #95059-154) was added for the colormetric analysis. An equal volume of 1 M phosphoric acid was added to quench the reaction, and OD₄₅₀ₙₘ was measured on the SpectraMax190 plate reader (Molecular Devices).

### (7) Confocal Microscope Images

The M13 phage was added to HeLa cells for 2 hours at 37° C. with 5% CO₂. To remove surface-bound phages, the cells were washed six times with cold PBS, three times with low pH glycine stripping buffer (50 mM glycine, 500 mM NaCl, pH=2.5), and two additional washes with PBS. The cells were fixed with 4% paraformaldehyde in PBS for 15 min at room temperature, followed by permeabilization with 0.1% Triton-X100 in PBS for 10 min at room temperature. The cells were then blocked for 30 min at 4° C. with 3% bovine serum albumin (BSA) in PBS. The cells were stained for 30 min at 4° C. with biotinylated M13 phage coat protein monoclonal antibody (Invitrogen, #MA1-12898) and anti-EGFR (BioLegend, #617501), followed by PBS washes. Subsequently, the cells were stained with streptavidin fluorescein-conjugate (BioLegend, #405204), anti-mouse IgG 650 (Abcam, #ab97018), and 4′,6-diamidino-2-phenylindole (DAPI) (BD Pharmingen, #564907) for 30 min at 4° C., followed by PBS washes. The samples were imaged by the confocal microscope Zeiss LSM780.

### (8) Transmission Electron Microscope Images

The PEG-purified M13 phage was further purified by biotinylated M13 phage coat protein monoclonal antibody (Invitrogen, #MA1-12898) immobilized on the streptavidin MagneSphere (Promega MagneSphere magnetic separation products, #Z5481). After capturing and washes, the phage was eluted by 0.1 M acetic acid and quickly neutralized by 1.0 M Tris-HCl buffer (pH=11). The purified phage was applied on the copper grids supported with thin carbon layer (EMS) and stained by 2% uranyl acetate. The images were taken by JEOL 1400 120-kV TEM with 4K×4K CCD camera (Gatan 895) under 20k× to 50k× magnification, and the defocus was about 1.5 μm.

### (9) Phage and AAV Transduction of Mammalian Cells

3×10⁴ target cells were seeded in 1 mL complete medium in the presence of 10% FBS in a 6-well plate. After 3 to 6 hours, the GFP-carrying phage in 100 L PBS was added to the cells. Depending on the experiments, an equal multiplicity of infection (MOI) (10⁶ or 10⁷ CFU/cell), an equal weight (0.13 ng/cell), or an equal production unit ( 1/10 of the phage production from 35 mL 2×YT culture) of the phage was applied. After 2 to 3 days, 2 mL of fresh complete medium was replenished. After 5 days, the GFP expression levels were analyzed by flow cytometry (Thermo Fisher, Attune NxT). The phage titer was determined by the E. coli infection efficiency (CFU) using a surrogate phage with the wild-type pIII. The titer of EGF-phage was then calculated based on the qPCR data of the encapsulating gene.

For AAV transduction, AAV6, AAV8, or AAV9 that carried the GFP gene was applied to the target cells at the MOI of 10⁶ or 10⁷. The GFP expression levels were determined after 5 days.

### (10) Camptothecin Treatment Experiments

The cells were treated with 10 μM camptothecin (CPT) for 7 hours and replaced with the complete medium for 48 hours before qPCR analysis. For phage transduction, the phage was added to the cells for 40 hours, and the cells were treated with 10 M camptothecin for 7 hours, followed by the replacement with the complete medium for 48 hours before the flow cytometry analysis^{[9]}.

### (11) RNA-Seq Experiments

The RNA was extracted from PrimPol-HeLa and PrimPol-HeLa^{hi} using RNeasy Mini Kit (QIAGEN, #74106). The purified RNA was used for the sequencing library preparation with the TruSeq Stranded mRNA Library Prep Kit (Illumina, San Diego, CA, USA) following the manufacturer's recommendations. Briefly, oligo(dT)-coupled magnetic beads were used to purify mRNA from total RNA (1 μg). The mRNA was fragmented into small pieces under elevated temperature. The first-strand cDNA was synthesized using reverse transcriptase and random primers. After the generation of double-strand cDNA and adenylation on 3′ ends of DNA fragments, the adaptors were ligated and purified with AMPure XP system (Beckman Coulter, Beverly, USA). The quality of the libraries was assessed on the Qsep 400 system. The qualified libraries were then sequenced on an Illumina NovaSeq 6000 platform with 150 base pairs (bp) paired-end reads generated by Genomics, BioSci & Tech Co., New Taipei City, Taiwan. The bases with low quality and sequences from adapters in raw data were removed using program fastp (version 0.20.0). The filtered reads were aligned to the reference genomes using HISAT2 (version 2.1.0). The software FeatureCounts (version 2.0.1) in Subread package was applied for the quantification of the gene abundance. Differentially expressed genes (DEGs) were identified by DESeq2 (version 1.28.0). The functional enrichment analysis of Gene Ontology (GO) terms was implemented in an R package clusterProfiler (version 4.0.0).

### (12) Generation of the DMBT1-Knockout (KO) Cell Line

The plasmid that carried the Cas9 from Streptococcus pyogenes (SpCas9), the sgRNA targeting the DMBT1 gene (protospacer: gcgagtggaggtcctataccg (SEQ ID NO. 9), the +1 position G was for the U6 promoter initiation), and the puromycin selection marker was transfected into the HeLa cells by Lipofectamine 2000 according to the manufacturer's protocol. After 2 days of transfection, the cells were transiently selected by puromycin (2 μg/mL) for 2 days. The survived cells were recovered in a puromycin-free medium for 1 week before further experiments.

### (13) Pyroptosis Induction Assay

2,000 target cells were seeded in a 96-well plate. The N-terminal GSDMD-carrying phage was added to the cells and incubated for 5 days at 37° C. with 5% CO₂. The cell viability was measured with cell counting Kit-8 (CCK-8, Dojindo Molecular Technologies) according to the manufacturer's instructions.

### (14) NK-Cell Killing Assay

1.5×10⁶ target cells were labeled with 1.5 mL of 10 μM Calcein-AM in PBS, incubated at 37° C. for 30 min, and then washed by 1 mL culture medium for 3 times. The cell number was adjusted to 1×10⁵ cells/mL. The labeled target cells were seeded into a U-bottom 96-well plate at a density of 1×10⁴ cells/100 μL/well, and the effector cells (NK-92 or NK-92 CD16′) were co-seeded at various E:T ratios. The plate was centrifuged at 120 g for 3 min to increase the contact of cells and incubated for 4 hours at 37° C. Subsequently, the plate was centrifuged at 120 g for 3 min, followed by transferring 100 μL supernatant to a 96-well plate.

The fluorescence signal was measured by Tecan-Infinite M1000 PRO microplate reader (Excitation=488; Emission=520). The percentage of cytotoxicity was calculated as:

$$Cytotoxicity ( \% ) = \frac{Experimental release - Spontaneous release}{Maximal release - Spontaneous release} \times 100 ,$$

where the spontaneous release and the maximal release were determined by no treatment and the lysis buffer (2% Triton X-100) treatment, respectively.

### (15) Mouse Experiments

Male nude mice were provided by the National Laboratory Animal Center (NLAC), NARLabs, Taiwan. All animal studies were conducted under specific pathogen-free conditions and in accordance with guidelines approved by the Animal Care and Usage Committee of Academia Sinica. 6- to 8-week-old mice were inoculated subcutaneously with 5×10⁶ PC3 cells that overexpressed PrimPol at the right flank. When the tumor volume reached 100 to 200 mm³, about 10¹² particles of EGF-TransPhage encapsulating mCherry, membrane-bound mouse Fc, or membrane-bound human Fc were intratumorally injected into the tumor every other day for four times. The tumor size was estimated by the formula [(Length+Width/2)]³×0.52.

### Example 1: Ligand-Displayed Phage was Highly Selective in Delivering the Gene of Interest into the Cells with High Receptor Expression

The advantage of the M13 phage as a gene delivery vehicle was that the infection tropism can be straightforwardly engineered by displaying a cell-targeting ligand on the pIII capsid protein. In order to achieve a highly specific transduction toward cancer cells, the vehicle is able to distinguish the cells with high receptor expression levels from the low-expressing ones because most tumor-associated antigens (TAAs) were also expressed in healthy cells at moderate levels.

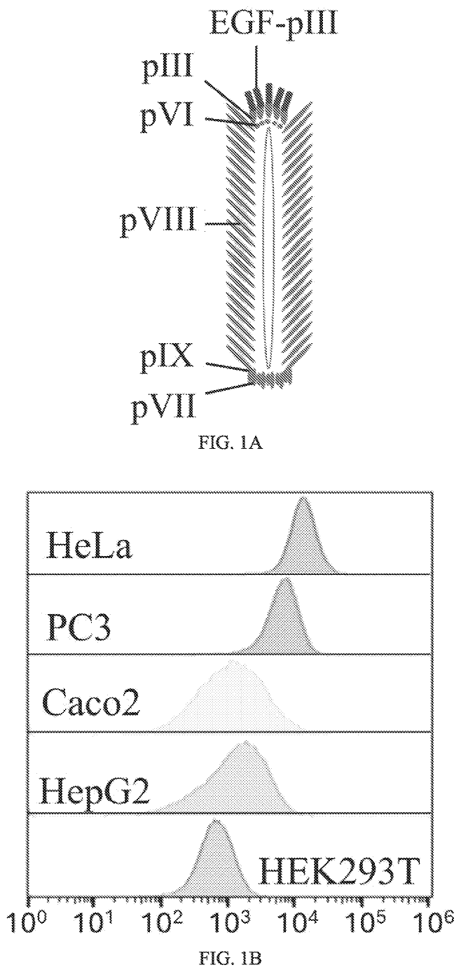

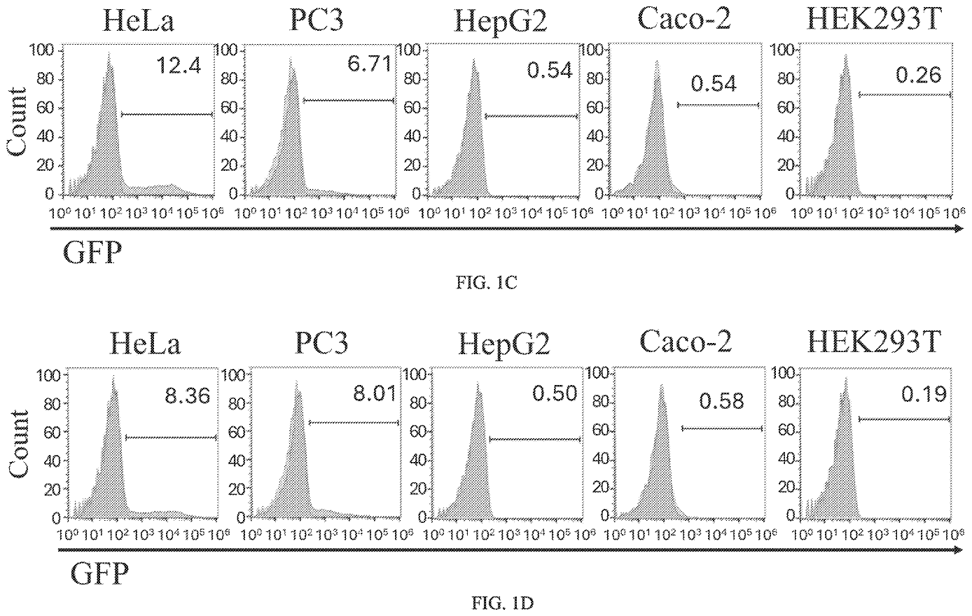

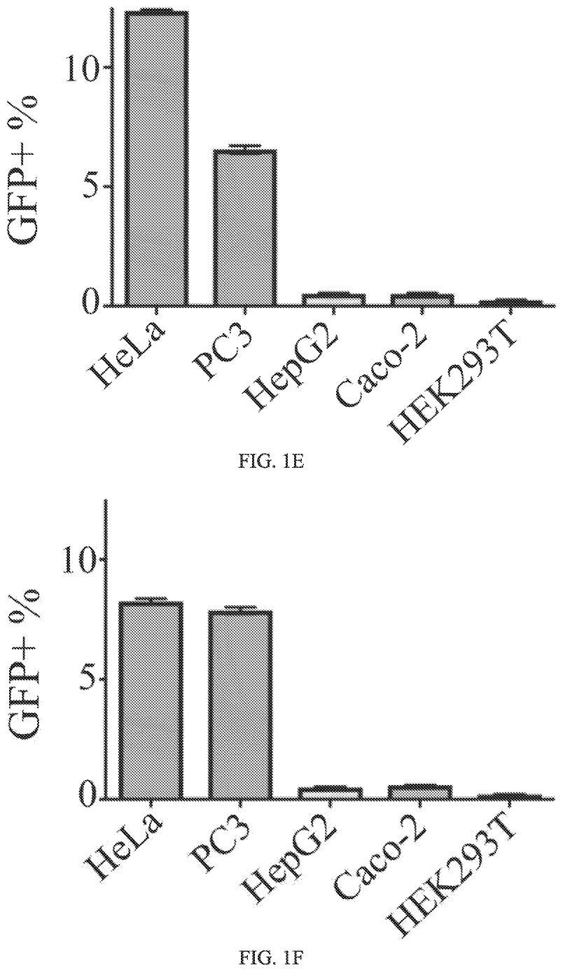

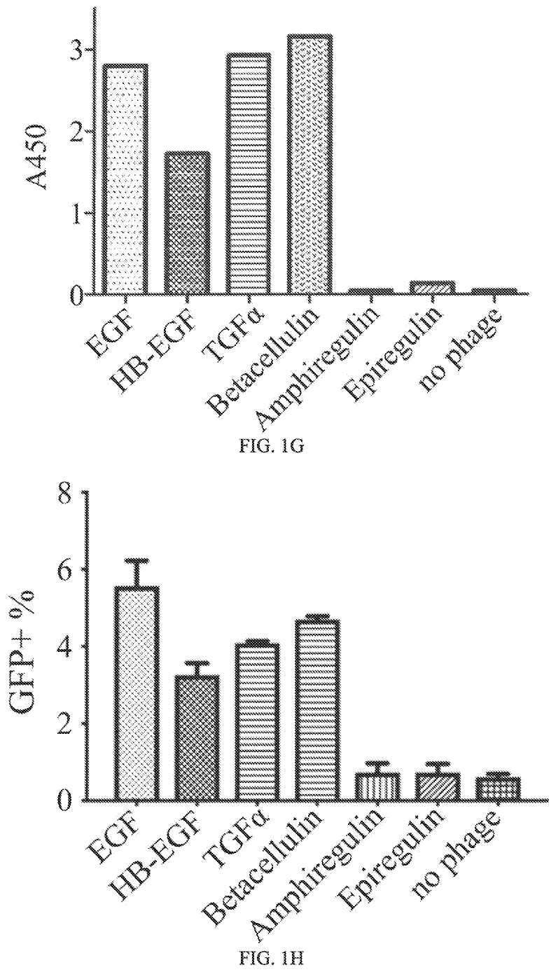

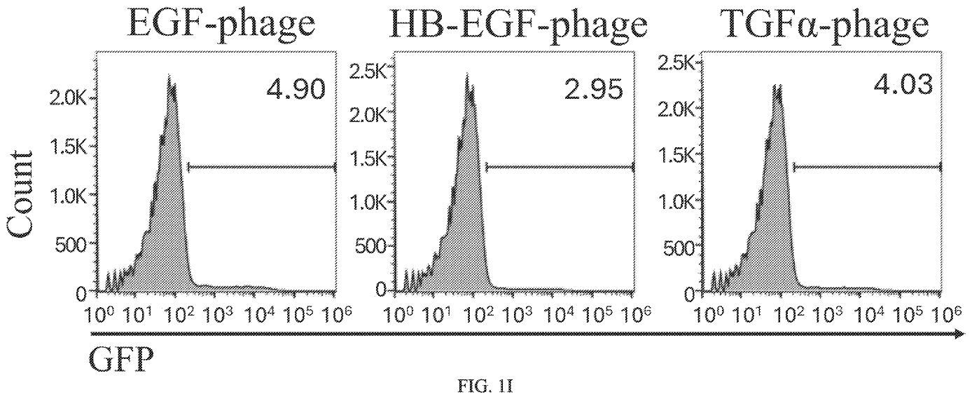

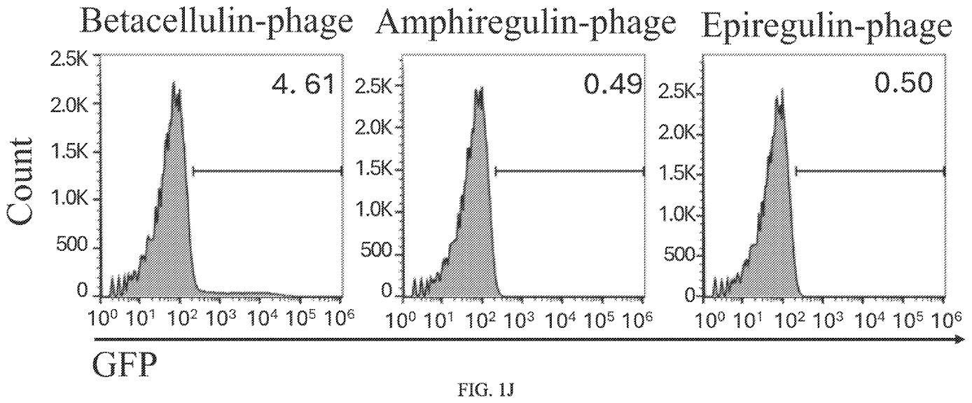

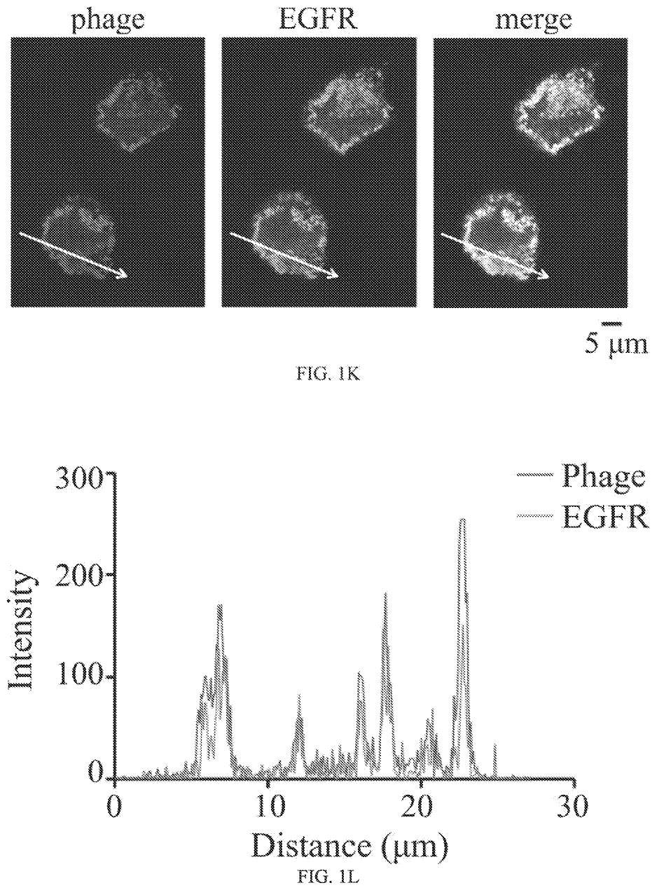

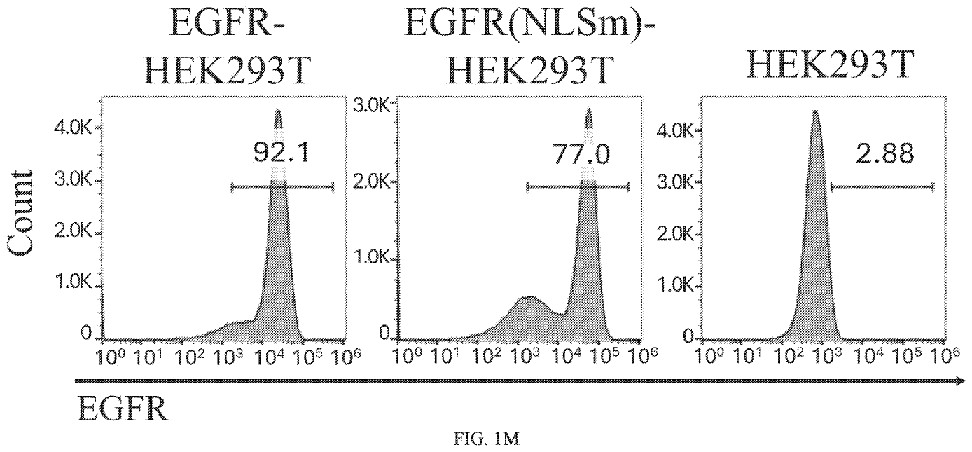

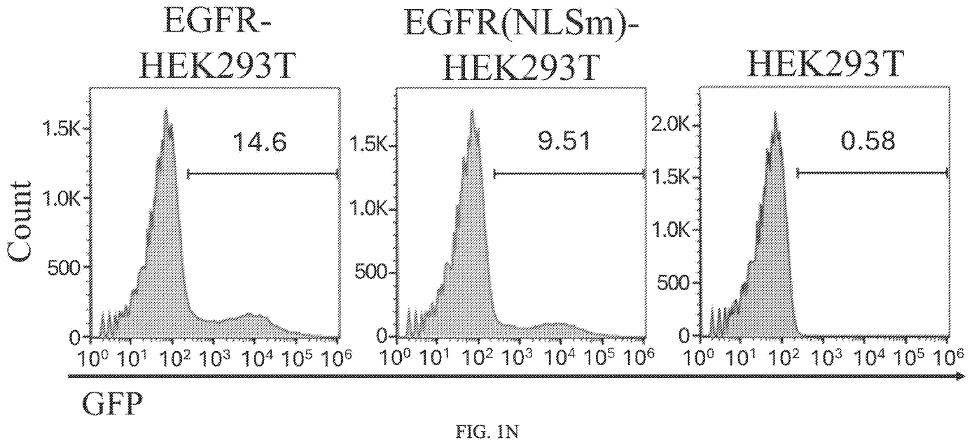

To investigate if the M13 phage had said favorable property, the epidermal growth factor (EGF) was displayed on pIII of M13 phage in a multivalent manner (named herein as EGF-phage^{6k}) (FIG. 1A), in which the phage was produced from the E. coli co-transformed with the helper plasmid and the phagemid. Further, the transduction efficiency of EGF-phage^{6k} over five human cell lines with various EGFR expression levels was evaluated (FIG. 1B). It was found that the EGF-phage^{6k} (the phagemid size was 6,133 nt) successfully delivered the GFP reporter gene into the two cell lines, HeLa and PC3, which highly expressed EGFR (FIGS. 1C and 1E). Further, the cell lines with moderate EGFR expression (HepG2 and Caco-2) or negligible EGFR expression (HEK293T) were refractory to the EGF-phage^{6k} transduction.

It has been reported that EGF and transforming growth factor-α (TGFα), both are high-affinity ligands of EGFR, undergo different intracellular trafficking upon the internalization with EGFR^{[10]}. Unlike EGF that causes the lysosomal degradation of EGFR, TGFα leads to the receptor recycling. Regarding this difference, however, it was found that TGFα-phage^{6k} and EGF-phage^{6k} exhibited similar transduction profiles over the five cell lines (FIGS. 1C to 1F).

Furthermore, by evaluating the displaying panel to all six natural ligands of EGFR, which have different binding affinities and trigger various endocytic sorting, it was found that the binding strengths, but not the sorting pathways, determined the phage transduction efficiency. For example, the four strong ligands (EGF, HB-EGF, TGFα, and betacellulin; Kd=about 0.1 nM to 1 nM) led to comparable transduction levels in the HeLa cells, whereas the two weak ligands (amphiregulin and epiregulin; Kd>10 nM) relatively failed to deliver the reporter gene (FIGS. 1G to 1J). Overall, these results revealed that the M13 phage transduction was modulated in a binary manner by the expression level of the target receptor and the binding strength of the displayed ligand.

To confirm that the gene delivery was truly mediated by EGFR, the immunostaining of the phage and EGFR in HeLa cells was performed, and the strong co-localization signals in the cells were observed. As shown in FIGS. 1K and 1L, the phage was co-internalized with EGFR. Additionally, through the engineered HEK293T cells with EGFR overexpression (FIG. 1M), it was confirmed that the EGFR-11EK293T cells became susceptible to the EGF-phage transduction (FIG. 1N).

To understand if the EGF-phage gene delivery was associated with the nuclear translocation property of EGFR, another HEK293T stable cell line was engineered to express an EGFR variant with the abrogating mutations in the nuclear localization signal (NLS). As shown in FIGS. 1M and 1N, said EGFR(NLSm)-HEK293T cell line only showed slightly decreased transduction efficiency compared to the wild-type EGFR-HEK293T cell line, especially after the normalization with the EGFR-positive population. These results indicated that the nuclear translocation of EGFR was not critical for the EGF-phage transduction.

### Example 2: PrimPol Up-Regulation Enhanced the Gene Delivery Efficiency of Phage

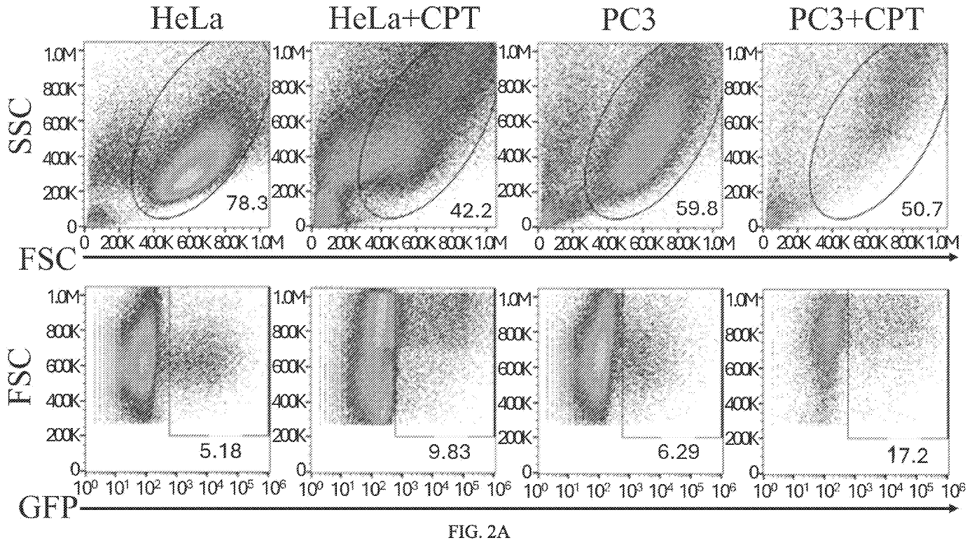

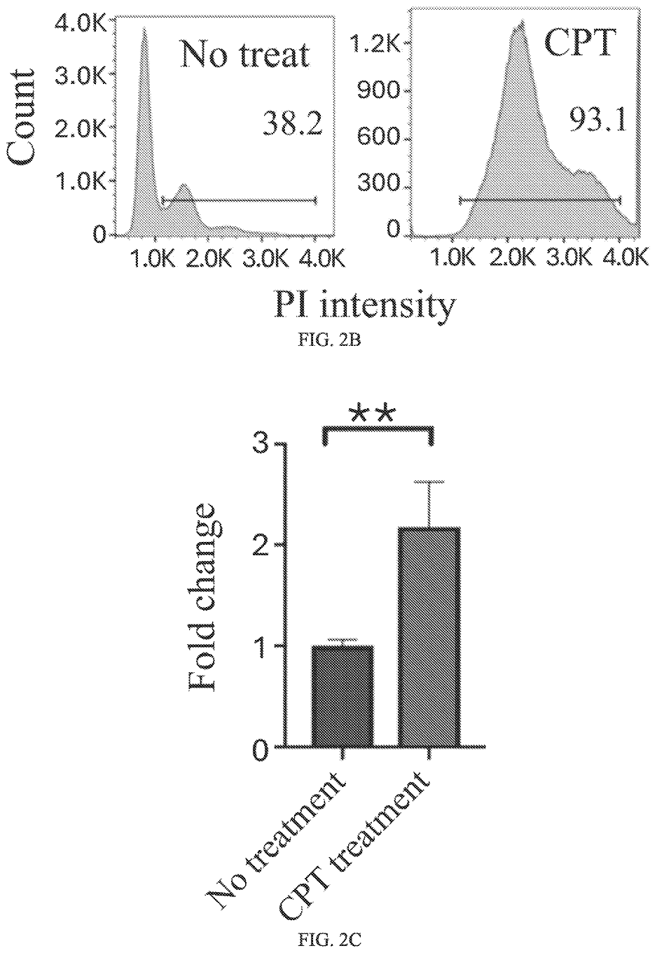

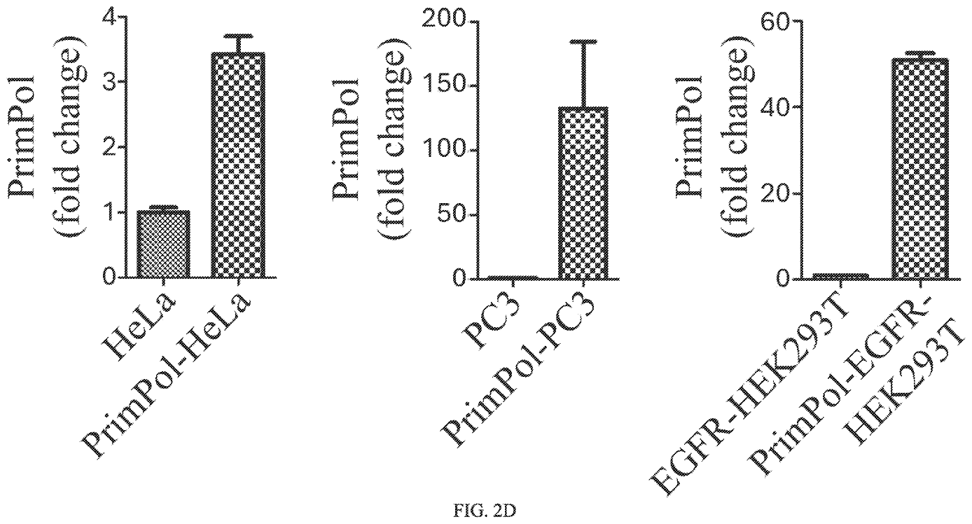

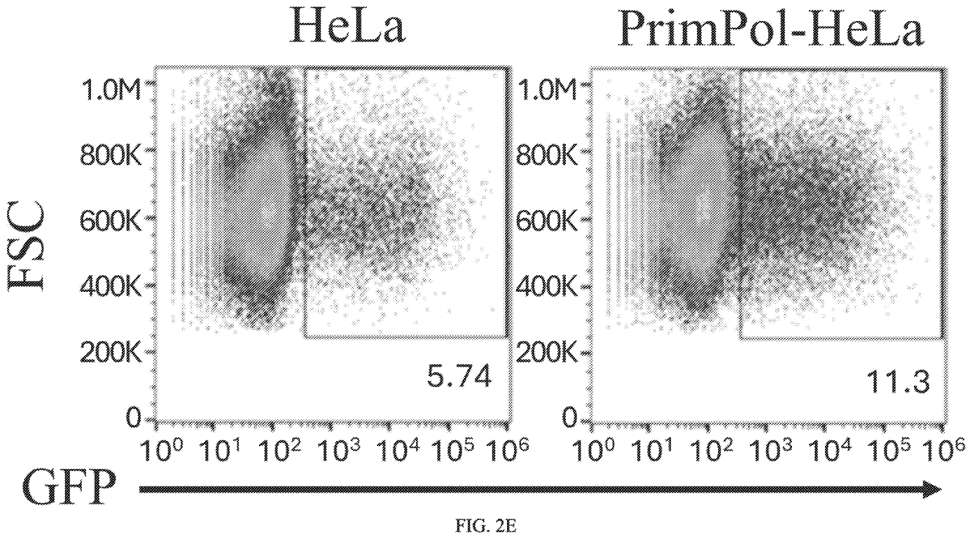

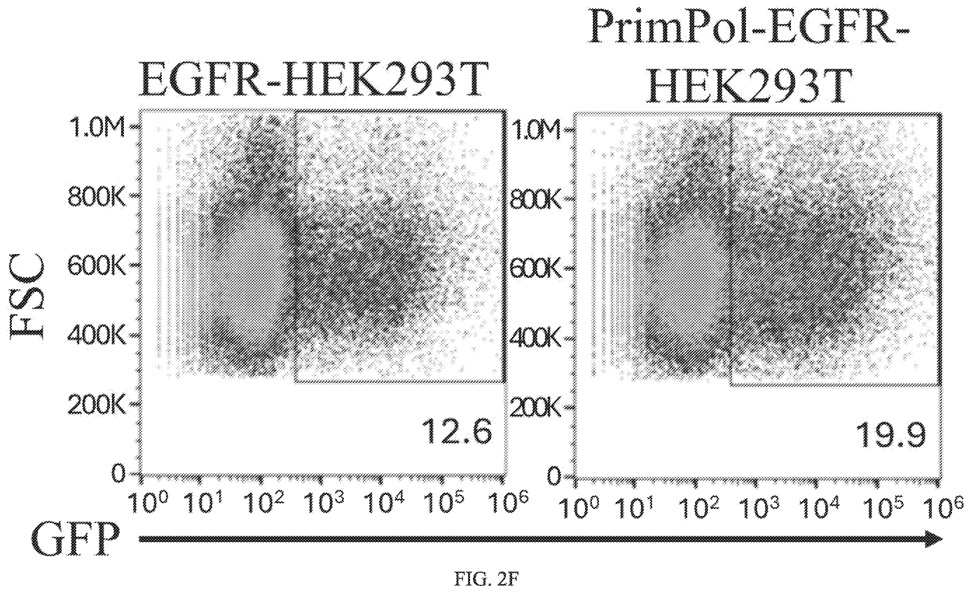

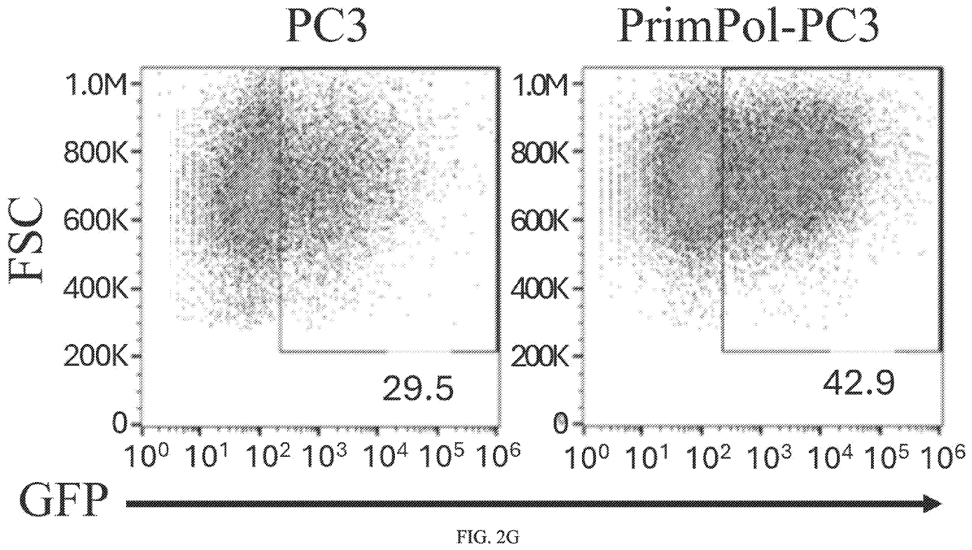

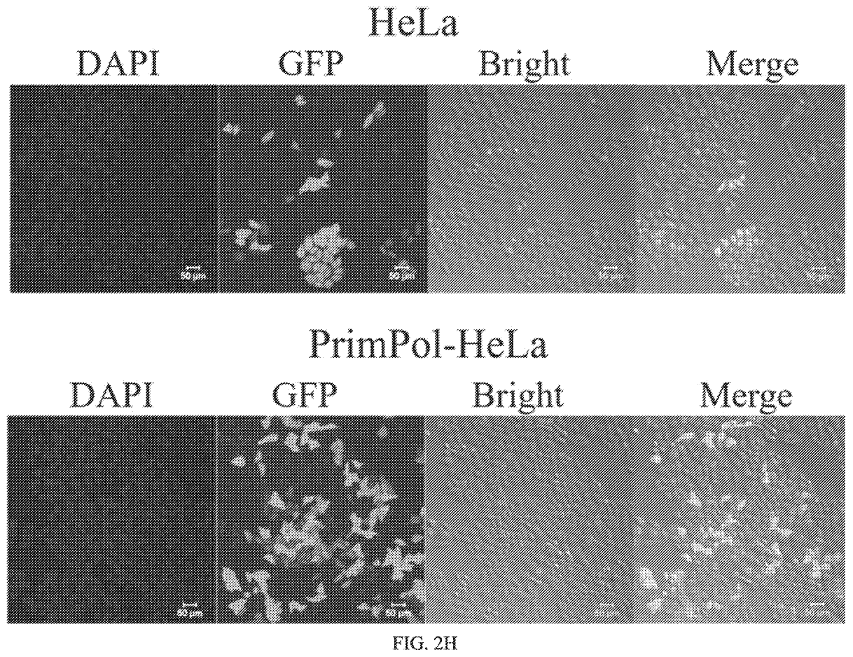

To investigate if the intracellular factors influenced the M13 transduction efficiency, the transduction of EGF-phage^{6k} (GFP) on HeLa and PC3 was performed in the presence or absence of camptothecin (CPT), a topoisomerase inhibitor commonly used for cancer chemotherapy. It was found that the CPT treatment greatly enhanced the transduction efficiency by 2 to 3 folds for both cell types (FIG. 2A). It was also noticed that the cell size and the granularity were enlarged by the CPT treatment as shown by flow cytometry. The cell cycle analysis revealed that CPT caused the cell cycle arrest at the G2/M phase (FIG. 2B), which led to a complete cessation of cell proliferation.

It has been reported that PrimPol, a human protein with dual activities of polymerase and primase, is involved in translesion synthesis (TLS) of DNA to bypass the damages on chromosomes^{[11]}. As shown in FIG. 2C, the PrimPol transcript in HeLa was up-regulated by the CPT treatment. To further investigate if PrimPol converted the phage-delivered single-stranded DNA (ssDNA) to double-stranded DNA (dsDNA) and promoted the transgene expression, a HeLa cell line overexpressing PrimPol was created (FIG. 2D), which exhibited about 2-fold improvement of the phage transduction efficiency (FIGS. 2E and 2H), similar to the effect of the CPT treatment without cell distortion or cycle arrest. Further, PrimPol-overexpressing cell lines in two other cell lines, PC3 and EGFR-HEK293T, were also created (FIG. 2D). Both cell lines showed enhanced phage transduction efficiency when compared to the parental cells (FIGS. 2F and 2G).

Overall, these results indicated that PrimPol was a general intracellular protein for enhancing the M13 phage transduction in human cells.

### Example 3: Transcriptomic Data Revealed DMBT1 as a Pathogen Recognition Protein that Sequestered the Phage Transduction

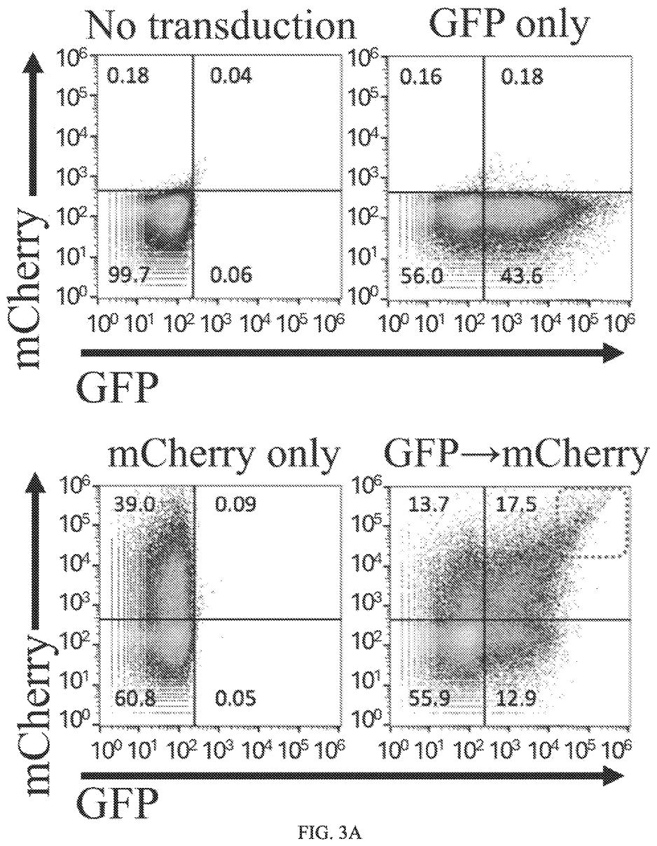

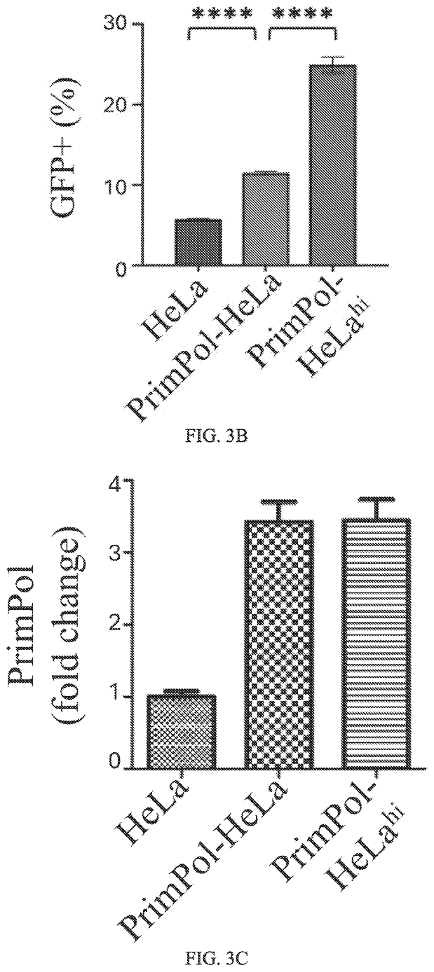

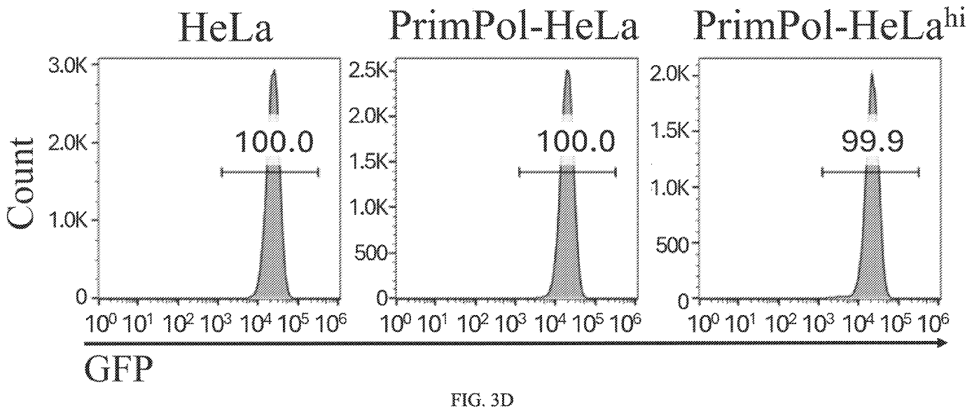

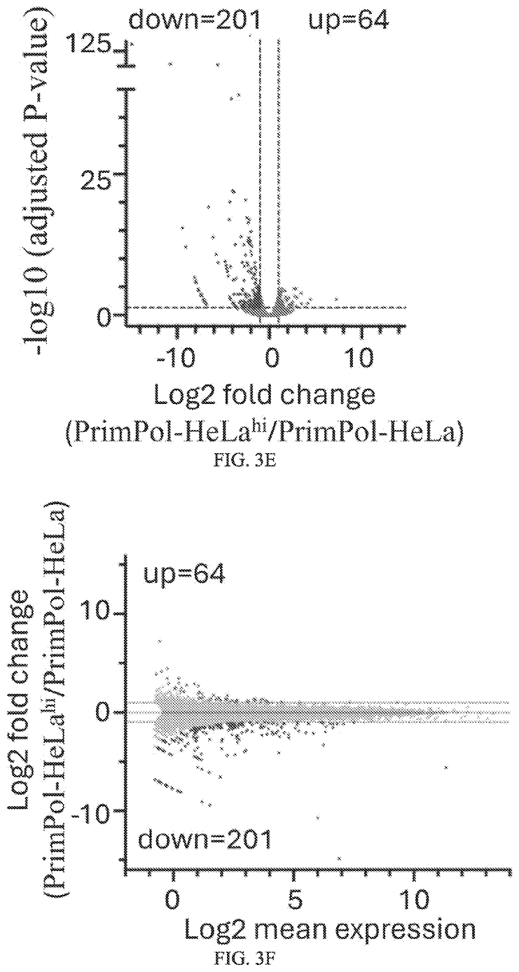

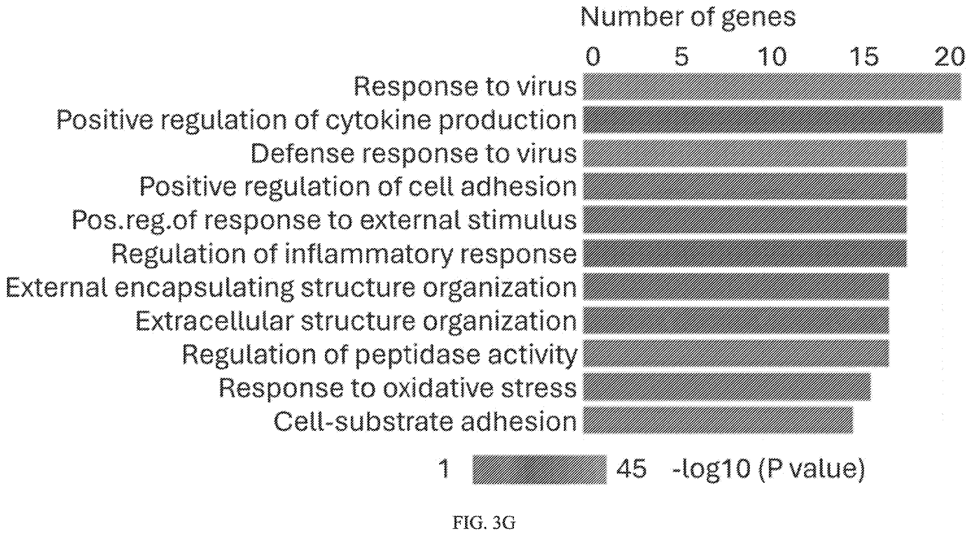

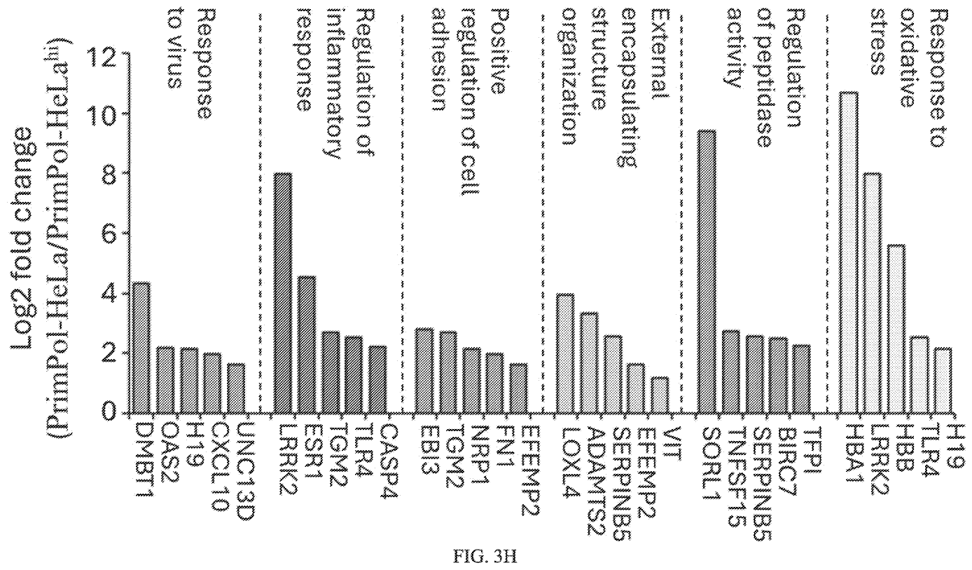

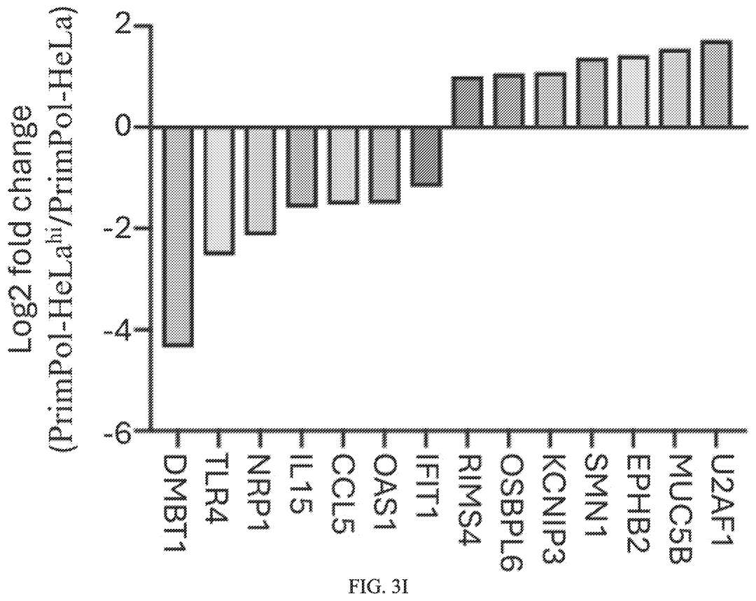

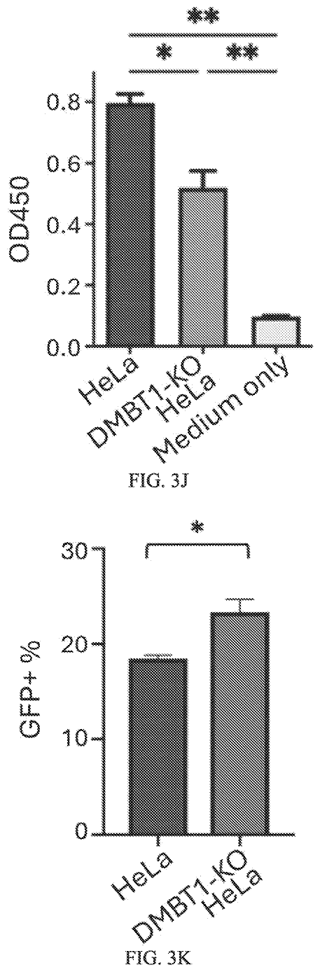

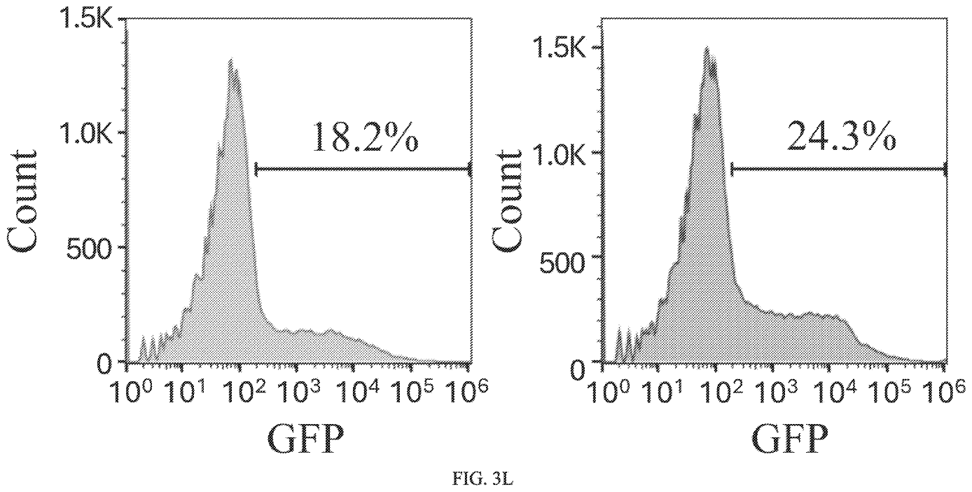

In the attempts to deliver multiple genes (GFP and mCherry) using successive transduction, it was found that a special population of PrimPol-HeLa was particularly susceptible to double infections with high expression levels (FIG. 3A). This population (PrimPol-HeLa^{hi}) was isolated by sorting, and it was observed that PrimPol-HeLa^{hi} exhibited a significantly higher transduction efficiency than the parental population (FIG. 3B). Further, FIGS. 3C and 3D showed the PrimPol and EGFR levels of PrimPol-HeLa and PrimPol-HeLa^{hi}, and there was no difference between these two cells.

By comparing the transcriptomes of PrimPol-HeLa and PrimPol-HeLa^{hi} using RNA-seq experiments, the fold-change analysis revealed a skewed distribution with many transcripts down-regulated in the PrimPol-HeLa^{hi} cells (FIGS. 3E and 3F, and Table 1). Also, the GO-term analysis revealed several terms that might shed light on the phage transduction process, including “response to virus,” “regulation of inflammatory response,” “external encapsulating structure organization,” “regulation of peptidase activity,” and the like (FIGS. 3G and 3H, and Table 2). For example, the Deleted in Malignant Brain Tumors 1 (DMBT1) was the most down-regulated protein (20.4 folds) in the “response to virus” term. The Ingenuity Pathway Analysis (IPA) predicted that the down-regulation of DMBT1 caused the increase of the viral infection in the PrimPol-HeLa^{hi} cells (FIG. 3I).

TABLE 1
Fold-change analysis of transcriptomes comparison between PrimPol-HeLa and PrimPol-HeLa^{hi}

| | | | | |
|---|---|---|---|---|
| | | | Adjusted | |
| | | Log2-fold | p-value | |
| Ensembl ID | Symbol | change | (padj) | Description |

Up Regulation

| | | | | |
|---|---|---|---|---|
| ENSG00000169469 | SPRR1B | 7.222693214 | 0.001560202 | small proline rich protein 1B |
| ENSG00000170465 | KRT6C | 4.45796828 | 0.001516199 | keratin 6C |
| ENSG00000242083 | RPL7AP31 | 4.154954661 | 0.009003183 | ribosomal protein L7a pseudogene 31 |
| ENSG00000189001 | SBSN | 3.87691502 | 0.036160076 | suprabasin |
| ENSG00000058866 | DGKG | 3.476674962 | 0.009019704 | diacylglycerol kinase gamma |
| ENSG00000165929 | TC2N | 3.456035657 | 0.000124347 | tandem C2 domains, nuclear |
| ENSG00000178184 | PARD6G | 3.006236901 | 0.000756589 | par-6 family cell polarity regulator gamma |
| ENSG00000260458 | KCNJ18 | 2.922535524 | 0.027010685 | potassium inwardly rectifying channel |
| | | | | subfamily J member 18 |
| ENSG00000107731 | UNC5B | 2.901547223 | 0.000020499 | unc-5 netrin receptor B |
| ENSG00000185479 | KRT6B | 2.757860005 | 0.000449815 | keratin 6B |
| ENSG00000186847 | KRT14 | 2.696711026 | 0.000019492 | keratin 14 |
| ENSG00000163220 | S100A9 | 2.487531655 | 0.004113491 | S100 calcium binding protein A9 |
| ENSG00000124102 | PI3 | 2.472375517 | 0.010133641 | peptidase inhibitor 3 |
| ENSG00000189190 | ZNF600 | 2.472375517 | 0.010133641 | zinc finger protein 600 |
| ENSG00000135324 | MRAP2 | 2.411272989 | 0.014815005 | melanocortin 2 receptor accessory protein 2 |
| ENSG00000227191 | TRGC2 | 2.167022348 | 0.018945503 | T cell receptor gamma constant 2 |
| ENSG00000151240 | DIP2C | 2.129197105 | 0.014443751 | disco interacting protein 2 homolog C |
| ENSG00000205420 | KRT6A | 2.114035275 | 0.000698546 | keratin 6A |
| ENSG00000115738 | ID2 | 1.91530246 | 0.003148908 | inhibitor of DNA binding 2 |
| ENSG00000152284 | TCF7L1 | 1.904527146 | 0.00157955 | transcription factor 7 like 1 |
| ENSG00000245848 | CEBPA | 1.758861464 | 0.000630837 | CCAAT enhancer binding protein alpha |
| ENSG00000160201 | U2Af1 | 1.728327551 | 0.000049605 | U2 small nuclear RNA auxiliary factor 1 |
| ENSG00000117983 | MUC5B | 1.556518192 | 0.015568133 | mucin 5B, oligomeric mucus/gel-forming |
| ENSG00000149131 | SERPING1 | 1.490389067 | 0.043301226 | serpin family G member 1 |
| ENSG00000214975 | PPIAP29 | 1.459652711 | 0.004521768 | peptidylprolyl isomerase A pseudogene 29 |
| ENSG00000168993 | CPLX1 | 1.436510764 | 0.04947508 | complexin 1 |
| ENSG00000133216 | EPHB2 | 1.421742024 | 0.006550978 | EPH receptor B2 |
| ENSG00000226049 | TLK2P1 | 1.412362665 | 0.013724875 | tousled like kinase 2 pseudogene 1 |
| ENSG00000197444 | OGDHL | 1.412181011 | 0.010007372 | oxoglutarate dehydrogenase L |
| ENSG00000184060 | ADAP2 | 1.400407961 | 0.002890658 | ArfGAP with dual PH domains 2 |
| ENSG00000115844 | DLX2 | 1.383864933 | 0.000048422 | distal-less homeobox 2 |
| ENSG00000101977 | MCF2 | 1.37726233 | 0.000062465 | MCF.2 cell line derived transforming sequence |
| ENSG00000172062 | SMN1 | 1.375514605 | 0.000019492 | survival of motor neuron 1, telomeric |
| ENSG00000140545 | MFGE8 | 1.363841592 | 0.003972517 | milk fat globule EGF and factor V/VIII |
| | | | | domain containing |
| ENSG00000165959 | CLMN | 1.324822586 | 0.021584806 | calmin |
| ENSG00000188064 | WNT7B | 1.321191755 | 0.000062093 | Wnt family member 7B |
| ENSG00000141052 | MYOCD | 1.319832154 | 0.00644976 | myocardin |
| ENSG00000154511 | DIPK1A | 1.301181429 | 0.007265449 | divergent protein kinase domain 1A |
| ENSG00000117016 | RIMS3 | 1.289221736 | 0.046759626 | regulating synaptic membrane exocytosis 3 |
| ENSG00000211689 | TARP | 1.286802435 | 0.000226851 | TCR gamma alternate reading frame |
| | | | | protein |
| ENSG00000164318 | EGFLAM | 1.2507853 | 0.002986613 | EGF like, fibronectin type III and laminin |
| | | | | G domains |
| ENSG00000233936 | N/A* | 1.2507853 | 0.002986613 | N/A |
| ENSG00000172986 | GXYLT2 | 1.239236748 | 0.003344114 | glucoside xylosyltransferase 2 |
| ENSG00000137267 | TUBB2A | 1.225815754 | 0.006710374 | tubulin beta 2A class IIa |
| ENSG00000107537 | PHYH | 1.219551971 | 0.023626656 | phytanoyl-CoA 2-hydroxylase |
| ENSG00000087258 | GNAO1 | 1.215770842 | 0.004521768 | G protein subunit alpha ol |
| ENSG00000173698 | ADGRG2 | 1.205881275 | 0.000875104 | adhesion G protein-coupled receptor G2 |
| ENSG00000183801 | OLFML1 | 1.196041558 | 0.000288424 | olfactomedin like 1 |
| ENSG00000178726 | THBD | 1.163159492 | 0.002986613 | thrombomodulin |
| ENSG00000138193 | PLCE1 | 1.150922606 | 0.003044092 | phospholipase C epsilon 1 |
| ENSG00000172594 | SMPDL3A | 1.123920283 | 0.011931182 | sphingomyelin phosphodiesterase acid like 3A |
| ENSG00000153898 | MCOLN2 | 1.103317779 | 0.001122912 | mucolipin TRP cation channel 2 |
| ENSG00000135842 | NIBAN1 | 1.101939423 | 0.014756482 | niban apoptosis regulator 1 |
| ENSG00000115041 | KCNIP3 | 1.087141071 | 0.031910296 | potassium voltage-gated channel interacting |
| | | | | protein 3 |
| ENSG00000184254 | ALDH1A3 | 1.067774598 | 0.000722034 | aldehyde dehydrogenase 1 family member A3 |
| ENSG00000079156 | OSBPL6 | 1.060392296 | 0.009003183 | oxysterol binding protein like 6 |
| ENSG00000179750 | APOBEC3B | 1.050436765 | 0.000935496 | apolipoprotein B mRNA editing enzyme |
| | | | | catalytic subunit 3B |
| ENSG00000185585 | OLFML2A | 1.042070626 | 0.000122537 | olfactomedin like 2A |
| ENSG00000134760 | DSG1 | 1.038177376 | 0.002560342 | desmoglein 1 |
| ENSG00000136943 | CTSV | 1.025500579 | 0.002339974 | cathepsin V |
| ENSG00000163132 | MSX1 | 1.02448291 | 0.036503094 | msh homeobox 1 |
| ENSG00000170345 | FOS | 1.023858644 | 0.00012781 | Fos proto-oncogene, AP-1 transcription |
| | | | | factor subunit |
| ENSG00000101098 | RIMS4 | 1.010626613 | 0.000756589 | regulating synaptic membrane exocytosis 4 |
| ENSG00000268388 | FENDRR | 1.010117778 | 0.00609018 | FOXfl adjacent non-coding |
| | | | | developmental regulatory RNA |

Down Regulation

| | | | | |
|---|---|---|---|---|
| ENSG00000221923 | ZNF880 | −14.8757314 | 4.66E−135 | zinc finger protein 880 |
| ENSG00000206172 | HBA1 | −10.70191011 | 1.75E−108 | hemoglobin subunit alpha 1 |
| ENSG00000137642 | SORL1 | −9.410199847 | 3.45E−16 | sortilin related receptor 1 |
| ENSG00000197629 | MPEG1 | −9.057151778 | 7.78E−13 | macrophage expressed 1 |
| ENSG00000205810 | KLRC3 | −8.099239796 | 2.00E−07 | killer cell lectin like receptor C3 |
| ENSG00000188906 | LRRK2 | −7.977713394 | 9.54E−07 | leucine rich repeat kinase 2 |
| ENSG00000188660 | CH507-42P11.6 | −7.977713394 | 9.54E−07 | uncharacterized CH507-42P11.6 |
| ENSG00000083844 | ZNF264 | −7.934818094 | 1.65434E−06 | zinc finger protein 264 |
| ENSG00000261761 | LINC02616 | −7.698831367 | 2.52005E−05 | long intergenic non-protein coding RNA 2616 |
| ENSG00000283646 | LINC02009 | −7.646621101 | 4.24531E−05 | long intergenic non-protein coding RNA 2009 |
| ENSG00000121797 | CCRL2 | −7.416547127 | 0.000199337 | C-C motif chemokine receptor like 2 |
| ENSG00000198538 | ZNF28 | −7.416547127 | 0.000199337 | zinc finger protein 28 |
| ENSG00000249306 | LINC01411 | −7.352800577 | 0.000324719 | long intergenic non-protein coding RNA 1411 |
| ENSG00000168143 | FAM83B | −7.352800577 | 0.000324719 | family with sequence similarity 83 member B |
| ENSG00000234444 | ZNF736 | −7.352800577 | 0.000324719 | zinc finger protein 736 |
| ENSG00000169740 | ZNF32 | −7.142689369 | 0.001560202 | zinc finger protein 32 |
| ENSG00000144468 | RHBDD1 | −7.065253948 | 0.002604338 | rhomboid domain containing 1 |
| ENSG00000180479 | ZNF571 | −7.065253948 | 0.002604338 | zinc finger protein 571 |
| ENSG00000189280 | GJB5 | −6.983425332 | 0.004127197 | gap junction protein beta 5 |
| ENSG00000198626 | RYR2 | −6.983425332 | 0.004127197 | ryanodine receptor 2 |
| ENSG00000258179 | N/A | −6.804372458 | 0.011299144 | N/A |
| ENSG00000198681 | MAGEA1 | −6.558244237 | 6.33E−20 | MAGE family member A1 |
| ENSG00000250337 | PURPL | −6.08040014 | 1.51E−14 | p53 upregulated regulator of p53 levels |
| ENSG00000205809 | KLRC2 | −5.754969386 | 1.52E−11 | killer cell lectin like receptor C2 |
| ENSG00000244734 | HBB | −5.580224754 | 1.66E−107 | hemoglobin subunit beta |
| ENSG00000116690 | PRG4 | −5.559817192 | 4.99E−10 | proteoglycan 4 |
| ENSG00000166503 | HDGFL3 | −4.789957001 | 3.01E−10 | HDGF like 3 |
| ENSG00000227467 | LINC01537 | −4.681653296 | 1.53E−09 | long intergenic non-protein coding RNA 1537 |
| ENSG00000102271 | KLHL4 | −4.612540436 | 5.42E−09 | kelch like family member 4 |
| ENSG00000091831 | ESR1 | −4.539949375 | 1.96E−08 | estrogen receptor 1 |
| ENSG00000253981 | ALG1L13P | −4.437104789 | 1.14E−07 | ALG1 like 13, pseudogene |
| ENSG00000187908 | DMBT1 | −4.347723618 | 1.10E−17 | deleted in malignant brain tumors 1 |
| ENSG00000159905 | ZNF221 | −4.310407545 | 0.000598908 | zinc finger protein 221 |
| ENSG00000185736 | ADARB2 | −4.278668786 | 7.96E−21 | adenosine deaminase RNA specific B2 |
| | | | | (inactive) |
| ENSG00000214694 | ARHGEF33 | −4.195553121 | 0.001516199 | Rho guanine nucleotide exchange factor 33 |
| ENSG00000250920 | N/A | −4.188940187 | 2.53E−09 | N/A |
| ENSG00000275993 | LOC102724428 | −4.09357886 | 4.55E−39 | salt inducible kinase 1B (putative) |
| ENSG00000145908 | ZNF300 | −3.98331204 | 7.54E−23 | zinc finger protein 300 |
| ENSG00000138131 | LOXL4 | −3.934136722 | 0.005942792 | lysyl oxidase like 4 |
| ENSG00000204604 | ZNF468 | −3.878438891 | 5.89E−07 | zinc finger protein 468 |
| ENSG00000286051 | MAGEA6-DT | −3.860646714 | 0.009003183 | MAGEA6 divergent transcript |
| ENSG00000285588 | N/A | −3.860646714 | 0.009003183 | N/A |
| ENSG00000223552 | CCR5AS | −3.824873229 | 1.41E−22 | CCR5 antisense RNA |
| ENSG00000229656 | ITGB1-DT | −3.813458058 | 0.000209386 | ITGB1 divergent transcript |
| ENSG00000115339 | GALNT3 | −3.701382677 | 0.022729675 | polypeptide N- |
| | | | | acetylgalactosaminyltransferase 3 |
| ENSG00000105141 | CASP14 | −3.701382677 | 0.022729675 | caspase 14 |
| ENSG00000134830 | C5AR2 | −3.658936931 | 2.86E−13 | complement C5a receptor 2 |
| ENSG00000234911 | TEX21P | −3.61463224 | 0.036160076 | testis expressed 21, pseudogene |
| ENSG00000270038 | N/A | −3.61463224 | 0.036160076 | N/A |
| ENSG00000267750 | RUNDC3A-AS1 | −3.61463224 | 0.036160076 | RUNDC3A antisense RNA 1 |
| ENSG00000146374 | RSPO3 | −3.604181331 | 3.19E−07 | R-spondin 3 |
| ENSG00000260896 | ARLNC1 | −3.410340954 | 0.000213144 | androgen receptor regulated long noncoding |
| | | | | RNA 1 |
| ENSG00000087116 | ADAMTS2 | −3.317113341 | 9.54E−40 | ADAM metallopeptidase with |
| | | | | thrombospondin type 1 motif 2 |
| ENSG00000240771 | ARHGEF25 | −3.210635146 | 0.006019069 | Rho guanine nucleotide exchange factor 25 |
| ENSG00000142556 | ZNF614 | −3.115192298 | 0.000208778 | zinc finger protein 614 |
| ENSG00000258927 | N/A | −3.080422742 | 0.003292492 | N/A |
| ENSG00000103534 | TMC5 | −3.050313926 | 1.21E−14 | transmembrane channel like 5 |
| ENSG00000076356 | PLXNA2 | −3.010267397 | 0.021734192 | plexin A2 |
| ENSG00000125962 | ARMCX5 | −3.010267397 | 0.021734192 | armadillo repeat containing X-linked 5 |
| ENSG00000118557 | PMFBP1 | −3.010267397 | 0.021734192 | polyamine modulated factor 1 binding protein 1 |
| ENSG00000229951 | FOSL2-AS1 | −2.936777388 | 0.03282089 | FOSL2 antisense RNA 1 |
| ENSG00000153253 | SCN3A | −2.936777388 | 0.03282089 | sodium voltage-gated channel alpha subunit 3 |
| ENSG00000165072 | MAMDC2 | −2.905541111 | 4.72E−10 | MAM domain containing 2 |
| ENSG00000231881 | N/A | −2.905223725 | 2.95269E−05 | N/A |
| ENSG00000188603 | CLN3 | −2.882043222 | 0.014815005 | CLN3 lysosomal/endosomal transmembrane |
| | | | | protein, battenin |
| ENSG00000172296 | SPTLC3 | −2.878324767 | 4.24531E−05 | serine palmitoyltransferase long chain base |
| | | | | subunit 3 |
| ENSG00000248668 | OXCT1-AS1 | −2.859341967 | 0.049286527 | OXCT1 antisense RNA 1 |
| ENSG00000279759 | N/A | −2.859341967 | 0.049286527 | N/A |
| ENSG00000142156 | COL6A1 | −2.827872321 | 0.021944997 | collagen type VI alpha 1 chain |
| ENSG00000105246 | EBI3 | −2.811428273 | 0.002890658 | Epstein-Barr virus induced 3 |
| ENSG00000079308 | TNS1 | −2.753122639 | 3.17E−13 | tensin 1 |
| ENSG00000181634 | TNFSf15 | −2.744980117 | 6.81E−07 | TNF superfamily member 15 |
| ENSG00000111700 | SLCO1B3 | −2.724323092 | 0.005944326 | solute carrier organic anion transporter family |
| | | | | member 1B3 |
| ENSG00000130827 | PLXNA3 | −2.713141212 | 2.25E−09 | plexin A3 |
| ENSG00000198959 | TGM2 | −2.713017897 | 0.046038842 | transglutaminase 2 |
| ENSG00000169752 | NRG4 | −2.6787155 | 0.008358161 | neuregulin 4 |
| ENSG00000179304 | FAM156B | −2.63238964 | 0.000158015 | family with sequence similarity 156 member B |
| ENSG00000141574 | SECTM1 | −2.575955994 | 0.000217323 | secreted and transmembrane 1 |
| ENSG00000206075 | SERPINB5 | −2.569064733 | 2.21E−11 | serpin family B member 5 |
| ENSG00000059804 | SLC2A3 | −2.549219259 | 3.63E−21 | solute carrier family 2 member 3 |
| ENSG00000136869 | TLR4 | −2.532546246 | 0.02434167 | toll like receptor 4 |
| ENSG00000101197 | BIRC7 | −2.496960863 | 0.000198531 | baculoviral IAP repeat containing 7 |
| ENSG00000204174 | NPY4R | −2.486937365 | 0.000631223 | neuropeptide Y receptor Y4 |
| ENSG00000169851 | PCDH7 | −2.425279143 | 5.13E−18 | protocadherin 7 |
| ENSG00000111181 | SLC6A12 | −2.416974397 | 4.95E−10 | solute carrier family 6 member 12 |
| ENSG00000073331 | ALPK1 | −2.399782447 | 0.005540485 | alpha kinase 1 |
| ENSG00000168899 | VAMP5 | −2.307127769 | 1.47E−09 | vesicle associated membrane protein 5 |
| ENSG00000119547 | ONECUT2 | −2.302502271 | 7.55E−14 | one cut homeobox 2 |
| ENSG00000248323 | LUCAT1 | −2.272672266 | 1.39E−08 | lung cancer associated transcript 1 |
| ENSG00000003436 | TFPI | −2.258683927 | 3.73E−17 | tissue factor pathway inhibitor |
| ENSG00000286966 | N/A | −2.239920349 | 0.014443751 | N/A |
| ENSG00000205609 | EIF3CL | −2.203956977 | 2.16E−14 | eukaryotic translation initiation factor 3 |
| | | | | subunit C like |
| ENSG00000196954 | CASP4 | −2.192345775 | 1.73E−13 | caspase 4 |
| ENSG00000111335 | OAS2 | −2.159827194 | 0.002143533 | 2′-5′-oligoadenylate synthetase 2 |
| ENSG00000081479 | LRP2 | −2.155078274 | 7.55E−14 | LDL receptor related protein 2 |
| ENSG00000196668 | LINC00173 | −2.142921654 | 0.015248495 | long intergenic non-protein coding RNA 173 |
| ENSG00000099250 | NRP1 | −2.136209921 | 0.000319854 | neuropilin 1 |
| ENSG00000130600 | H19 | −2.131011602 | 1.18E−17 | H19 imprinted maternally expressed transcript |
| ENSG00000173480 | ZNF417 | −2.129539897 | 0.002851597 | zinc finger protein 417 |
| ENSG00000116652 | DLEU2L | −2.107207576 | 0.038082592 | deleted in lymphocytic leukemia 2 like |
| ENSG00000234477 | N/A | −2.107207576 | 0.038082592 | N/A |
| ENSG00000221867 | MAGEA3 | −2.066182929 | 2.86E−13 | MAGE family member A3 |
| ENSG00000165124 | SVEP1 | −2.039715146 | 7.55E−14 | sushi, von Willebrand factor type A, EGF |
| | | | | and pontraxin domain containing 1 |
| ENSG00000185261 | KIAA0825 | −2.033841553 | 2.38E−08 | KIAA0825 |
| ENSG00000181577 | LINC03040 | −2.023140831 | 1.63E−15 | long intergenic non-protein coding RNA 3040 |
| ENSG00000179046 | TRIML2 | −2.009638906 | 0.004026483 | tripartite motif family like 2 |
| ENSG00000023839 | ABCC2 | −1.990401142 | 4.91E−12 | ATP binding cassette subfamily C member 2 |
| ENSG00000169245 | CXCL10 | −1.979813417 | 0.001663027 | C-X-C motif chemokine ligand 10 |
| ENSG00000115414 | FN1 | −1.951839355 | 1.49E−15 | fibronectin 1 |
| ENSG00000287974 | N/A | −1.929574192 | 5.54E−09 | N/A |
| ENSG00000129521 | EGLN3 | −1.926366285 | 3.15E−08 | egl-9 family hypoxia inducible factor 3 |
| ENSG00000214514 | KRT42P | −1.903286433 | 0.005670621 | keratin 42, pseudogene |
| ENSG00000250305 | TRMT9B | −1.897604295 | 0.022115675 | tRNA methyltransferase 9B (putative) |
| ENSG00000147027 | TMEM47 | −1.87795196 | 2.21E−11 | transmembrane protein 47 |
| ENSG00000109472 | CPE | −1.868614941 | 5.85E−13 | carboxypeptidase E |
| ENSG00000248019 | FAM13A-AS1 | −1.817573521 | 0.022204604 | FAM13A antisense RNA 1 |
| ENSG00000249992 | TMEM158 | −1.81239783 | 0.012868244 | transmembrane protein 158 |
| ENSG00000101213 | PTK6 | −1.789817687 | 2.82E−07 | protein tyrosine kinase 6 |
| ENSG00000184226 | PCDH9 | −1.789680095 | 3.88973E−05 | protocadherin 9 |
| ENSG00000116701 | NCF2 | −1.78078307 | 0.016459447 | neutrophil cytosolic factor 2 |
| ENSG00000125531 | FNDC11 | −1.747416006 | 0.038261004 | fibronectin type III domain containing 11 |
| ENSG00000099337 | KCNK6 | −1.722127878 | 0.021734192 | potassium two pore domain channel |
| | | | | subfamily K member 6 |
| ENSG00000213513 | IMPDH1P5 | −1.704813209 | 4.13851E−05 | inosine monophosphate dehydrogenase 1 |
| | | | | pseudogene 5 |
| ENSG00000285644 | N/A | −1.646713815 | 0.002604771 | N/A |
| ENSG00000172638 | EFEMP2 | −1.624169035 | 0.036883461 | EGF containing fibulin extracellular matrix |
| | | | | protein 2 |
| ENSG00000092929 | UNC13D | −1.62276401 | 7.12E−08 | unc-13 homolog D |
| ENSG00000109321 | AREG | −1.616113305 | 0.033983667 | amphiregulin |
| ENSG00000274487 | NPEPPSP1 | −1.616113305 | 0.033983667 | NPEPPS pseudogene 1 |
| ENSG00000227630 | LINC01132 | −1.604616505 | 0.045267394 | long intergenic non-protein coding RNA 1132 |
| ENSG00000231249 | ITPR1-DT | −1.59110511 | 0.04811367 | ITPR1 divergent transcript |
| ENSG00000286733 | LINC02348 | −1.59110511 | 0.04811367 | long intergenic non-protein coding RNA 2348 |
| ENSG00000164136 | IL15 | −1.587620562 | 0.043301226 | interleukin 15 |
| ENSG00000182585 | EPGN | −1.583580598 | 2.98E−09 | epithelial mitogen |
| ENSG00000254535 | PABPC4L | −1.579281695 | 0.000158015 | poly(A) binding protein cytoplasmic 4 like |
| ENSG00000179776 | CDH5 | −1.57196572 | 0.015248495 | cadherin 5 |
| ENSG00000187796 | CARD9 | −1.568679696 | 0.00014296 | caspase recruitment domain family |
| | | | | member 9 |
| ENSG00000228799 | LINC01939 | −1.563296924 | 0.000352959 | long intergenic non-protein coding RNA 1939 |
| ENSG00000188321 | ZNF559 | −1.562432175 | 0.001509481 | zinc finger protein 559 |
| ENSG00000250461 | N/A | −1.560878265 | 0.007487392 | N/A |
| ENSG00000131746 | TNS4 | −1.542040957 | 4.83E−08 | tensin 4 |
| ENSG00000178467 | P4HTM | −1.537085089 | 5.27E−10 | prolyl 4-hydroxylase, transmembrane |
| ENSG00000172159 | FRMD3 | −1.533589437 | 0.031910296 | FERM domain containing 3 |
| ENSG00000137878 | GCOM1 | −1.532726306 | 0.002168484 | GCOM1, MYZAP-POLR2M combined |
| | | | | locus |
| ENSG00000271503 | CCL5 | −1.527835121 | 1.90142E−06 | C-C motif chemokine ligand 5 |
| ENSG00000130818 | ZNF426 | −1.526635535 | 0.001785184 | zinc finger protein 426 |
| ENSG00000237940 | LINC01238 | −1.523168142 | 0.02434167 | long intergenic non-protein coding RNA 1238 |
| ENSG00000132470 | ITGB4 | −1.516787544 | 2.47066E−06 | integrin subunit beta 4 |
| ENSG00000089127 | OAS1 | −1.512008868 | 0.002168484 | 2′-5′-oligoadenylate synthetase 1 |
| ENSG00000181409 | AATK | −1.497380496 | 0.004559119 | apoptosis associated tyrosine kinase |
| ENSG00000196730 | DAPK1 | −1.475591306 | 0.000257125 | death associated protein kinase 1 |
| ENSG00000114646 | CSPG5 | −1.459666714 | 0.013037953 | chondroitin sulfate proteoglycan 5 |
| ENSG00000164171 | ITGA2 | −1.449223202 | 9.27E−09 | integrin subunit alpha 2 |
| ENSG00000137628 | DDX60 | −1.447864845 | 4.35E−07 | DExD/H-box helicase 60 |
| ENSG00000196793 | ZNF239 | −1.436564862 | 0.005401308 | zinc finger protein 239 |
| ENSG00000189350 | TOGARAM2 | −1.427811233 | 0.021734192 | TOG array regulator of axonemal |
| | | | | microtubules 2 |
| ENSG00000178038 | ALS2CL | −1.413150198 | 1.24349E−05 | ALS2 C-terminal like |
| ENSG00000260793 | ATXN7L3-AS1 | −1.41308158 | 0.000875882 | ATXN7L3 antisense RNA 1 |
| ENSG00000073756 | PTGS2 | −1.41013955 | 0.001237933 | prostaglandin-endoperoxide synthase 2 |
| ENSG00000243649 | CFB | −1.377859964 | 0.002800485 | complement factor B |
| ENSG00000135318 | NT5E | −1.358201486 | 0.000498717 | 5′-nucleotidase ecto |
| ENSG00000100342 | APOL1 | −1.340371512 | 0.016042013 | apolipoprotein L1 |
| ENSG00000073464 | CLCN4 | −1.320724409 | 0.002877601 | chloride voltage-gated channel 4 |
| ENSG00000266173 | STRADA | −1.304231461 | 0.029229693 | STE20 related adaptor alpha |
| ENSG00000249395 | CASC9 | −1.267014591 | 0.002127931 | cancer susceptibility 9 |
| ENSG00000185101 | ANO9 | −1.264980459 | 0.005944326 | anoctamin 9 |
| ENSG00000162804 | SNED1 | −1.258967548 | 0.000201914 | sushi, nidogen and EGF like domains 1 |
| ENSG00000160471 | COX6B2 | −1.230691008 | 6.16844E−05 | cytochrome c oxidase subunit 6B2 |
| ENSG00000167779 | IGFBP6 | −1.223977863 | 0.03616206 | insulin like growth factor binding protein 6 |
| ENSG00000170949 | ZNf160 | −1.222003848 | 0.000910852 | zinc finger protein 160 |
| ENSG00000143847 | PPFIA4 | −1.221054038 | 1.57119E−05 | PTPRF interacting protein alpha 4 |
| ENSG00000262877 | N/A | −1.20360433 | 0.031910296 | N/A |
| ENSG00000139211 | AMIGO2 | −1.197885316 | 0.005944326 | adhesion molecule with Ig like domain 2 |
| ENSG00000205221 | VIT | −1.178551456 | 0.006146678 | vitrin |
| ENSG00000185745 | IFIT1 | −1.177989419 | 6.30555E−06 | interferon induced protein with |
| | | | | tetratricopeptide repeats 1 |
| ENSG00000120708 | TGFBI | −1.176251315 | 7.51146E−06 | transforming growth factor beta induced |
| ENSG00000135046 | ANXA1 | −1.175918609 | 8.29488E−06 | annexin A1 |
| ENSG00000272668 | LOC107985216 | −1.172401907 | 0.007265449 | uncharacterized LOC107985216 |
| ENSG00000147689 | FAM83A | −1.162482072 | 0.000146329 | family with sequence similarity 83 member A |
| ENSG00000137819 | PAQR5 | −1.152129634 | 0.000146748 | progestin and adipoQ receptor family member 5 |
| ENSG00000197182 | MIRLET7BHG | −1.149404208 | 0.016159735 | MIRLET7B host gene |
| ENSG00000237187 | NR2f1-AS1 | −1.145863615 | 0.02434167 | NR2f1 antisense RNA 1 |
| ENSG00000120875 | DUSP4 | −1.143782904 | 4.4244E−05 | dual specificity phosphatase 4 |
| ENSG00000260261 | N/A | −1.133623713 | 0.01441063 | N/A |
| ENSG00000123405 | NFE2 | −1.130395906 | 0.022602469 | nuclear factor, erythroid 2 |
| ENSG00000149380 | P4HA3 | −1.122619983 | 0.009003183 | prolyl 4-hydroxylase subunit alpha 3 |
| ENSG00000001617 | SEMA3F | −1.122520884 | 0.000698546 | semaphorin 3F |
| ENSG00000172379 | ARNT2 | −1.106551095 | 4.39845E−05 | aryl hydrocarbon receptor nuclear translocator 2 |
| ENSG00000174652 | ZNF266 | −1.092423485 | 0.000927686 | zinc finger protein 266 |
| ENSG00000147676 | MAL2 | −1.091179661 | 7.90257E−05 | mal, T cell differentiation protein 2 |
| ENSG00000231770 | TMEM44-AS1 | −1.091051523 | 0.03212053 | TMEM44 antisense RNA 1 |
| ENSG00000144810 | COL8A1 | −1.080291551 | 0.001516199 | collagen type VIII alpha 1 chain |
| ENSG00000261051 | N/A | −1.079013654 | 0.04580995 | N/A |
| ENSG00000270012 | N/A | −1.078833886 | 0.049286527 | N/A |
| ENSG00000143382 | ADAMTSL4 | −1.068918375 | 0.00012781 | ADAMTS like 4 |
| ENSG00000119922 | IFIT2 | −1.041689727 | 0.000121241 | interferon induced protein with |
| | | | | tetratricopeptide repeats 2 |
| ENSG00000169994 | MYO7B | −1.032611524 | 0.006410186 | myosin VIIB |
| ENSG00000082497 | SERTAD4 | −1.020699715 | 0.000761853 | SERTA domain containing 4 |
| ENSG00000214029 | ZNF891 | −1.017097004 | 0.025233262 | zinc finger protein 891 |
| ENSG00000227964 | LINC01429 | −1.011224035 | 0.014443751 | long intergenic non-protein coding RNA 1429 |
| ENSG00000110002 | VWA5A | −1.010012087 | 0.000401451 | von Willebrand factor A domain containing 5A |
| ENSG00000127507 | ADGRE2 | −1.002280183 | 0.005994954 | adhesion G protein-coupled receptor E2 |

*N/A: Not applicable

TABLE 2
GO−term analysis of transcriptomes comparison between
PrimPol-HeLa and PrimPol-HeLa^{hi}

| | | | | | Adjusted |
|---|---|---|---|---|---|
| | | | | | p-value |
| ID | Description | Count | GeneRatio | BgRatio | (padj) |
| GO:0009615 | response to virus | 21 | 21/269 | 367/18723 | 0.00009783 |
| GO:0001819 | positive regulation of cytokine | 20 | 20/269 | 467/18723 | 0.00296984 |
| | production | | | | |
| GO:0051607 | defense response to virus | 18 | 18/269 | 265/18723 | 0.00009783 |
| GO:0140546 | defense response to symbiont | 18 | 18/269 | 265/18723 | 0.00009783 |
| GO:0050727 | regulation of inflammatory | 18 | 18/269 | 386/18723 | 0.00293591 |
| | response | | | | |
| GO:0032103 | positive regulation of response to | 18 | 18/269 | 427/18723 | 0.00659515 |
| | external stimulus | | | | |
| GO:0048732 | gland development | 18 | 18/269 | 436/18723 | 0.00701583 |
| GO:0045785 | positive regulation of cell adhesion | 18 | 18/269 | 437/18723 | 0.00701583 |
| GO:0030198 | extracellular matrix organization | 17 | 17/269 | 301/18723 | 0.00087167 |
| GO:0043062 | extracellular structure organization | 17 | 17/269 | 302/18723 | 0.00087167 |
| GO:0045229 | external encapsulating structure | 17 | 17/269 | 304/18723 | 0.00087167 |
| | organization | | | | |
| GO:0052547 | regulation of peptidase activity | 17 | 17/269 | 461/18723 | 0.01899947 |
| GO:0052548 | regulation of endopeptidase activity | 16 | 16/269 | 432/18723 | 0.02282698 |
| GO:0051960 | regulation of nervous system | 16 | 16/269 | 443/18723 | 0.02689121 |
| | development | | | | |
| GO:0006979 | response to oxidative stress | 16 | 16/269 | 446/18723 | 0.02795646 |
| GO:0031589 | cell-substrate adhesion | 15 | 15/269 | 363/18723 | 0.0149786 |
| GO:0050767 | regulation of neurogenesis | 15 | 15/269 | 364/18723 | 0.01506318 |
| GO:0042060 | wound healing | 15 | 15/269 | 422/18723 | 0.03687443 |
| GO:0010720 | positive regulation of cell | 14 | 14/269 | 298/18723 | 0.00917146 |
| | development | | | | |
| GO:0007517 | muscle organ development | 14 | 14/269 | 327/18723 | 0.015648 |
| GO:0048545 | response to steroid hormone | 14 | 14/269 | 339/18723 | 0.01957287 |
| GO:0030900 | forebrain development | 14 | 14/269 | 379/18723 | 0.03687443 |
| GO:0031349 | positive regulation of defense | 13 | 13/269 | 278/18723 | 0.01241135 |
| | response | | | | |
| GO:1901214 | regulation of neuron death | 13 | 13/269 | 319/18723 | 0.02768835 |
| GO:0002831 | regulation of response to biotic | 13 | 13/269 | 327/18723 | 0.03096394 |
| | stimulus | | | | |
| GO:0045861 | negative regulation of proteolysis | 13 | 13/269 | 351/18723 | 0.0502262 |
| GO:0006887 | exocytosis | 13 | 13/269 | 352/18723 | 0.0502262 |
| GO:0048660 | regulation of smooth muscle cell | 12 | 12/269 | 180/18723 | 0.00287238 |
| | proliferation | | | | |
| GO:0048659 | smooth muscle cell proliferation | 12 | 12/269 | 184/18723 | 0.00296984 |
| GO:0010810 | regulation of cell-substrate adhesion | 12 | 12/269 | 221/18723 | 0.00806699 |
| GO:0000302 | response to reactive oxygen species | 12 | 12/269 | 222/18723 | 0.00806699 |
| GO:0007599 | hemostasis | 12 | 12/269 | 222/18723 | 0.00806699 |
| GO:0033002 | muscle cell proliferation | 12 | 12/269 | 248/18723 | 0.0149786 |
| GO:0051962 | positive regulation of nervous | 12 | 12/269 | 272/18723 | 0.02329639 |
| | system development | | | | |
| GO:0032640 | tumor necrosis factor production | 11 | 11/269 | 181/18723 | 0.00701583 |
| GO:0032680 | regulation of tumor necrosis factor | 11 | 11/269 | 181/18723 | 0.00701583 |
| | production | | | | |
| GO:0071706 | tumor necrosis factor superfamily | 11 | 11/269 | 186/18723 | 0.00806699 |
| | cytokine production | | | | |
| GO:1903555 | regulation of tumor necrosis factor | 11 | 11/269 | 186/18723 | 0.00806699 |
| | superfamily cytokine production | | | | |
| GO:0017157 | regulation of exocytosis | 11 | 11/269 | 202/18723 | 0.01129431 |
| GO:0007596 | blood coagulation | 11 | 11/269 | 217/18723 | 0.01645474 |
| GO:0050817 | coagulation | 11 | 11/269 | 222/18723 | 0.01899947 |
| GO:0050769 | positive regulation of neurogenesis | 11 | 11/269 | 225/18723 | 0.01957287 |
| GO:0045055 | regulated exocytosis | 11 | 11/269 | 230/18723 | 0.02185815 |
| GO:0010951 | negative regulation of | 11 | 11/269 | 252/18723 | 0.03461849 |
| | endopeptidase activity | | | | |
| GO:0010466 | negative regulation of peptidase | 11 | 11/269 | 262/18723 | 0.043775 |
| | activity | | | | |
| GO:0098586 | cellular response to virus | 10 | 10/269 | 84/18723 | 0.00029158 |
| GO:0032760 | positive regulation of tumor | 10 | 10/269 | 103/18723 | 0.00087167 |
| | necrosis factor production | | | | |
| GO:1903557 | positive regulation of tumor | 10 | 10/269 | 107/18723 | 0.00111111 |
| | necrosis factor superfamily | | | | |
| | cytokine production | | | | |
| GO:0010811 | positive regulation of cell-substrate | 10 | 10/269 | 123/18723 | 0.00287238 |
| | adhesion | | | | |
| GO:0050729 | positive regulation of inflammatory | 10 | 10/269 | 142/18723 | 0.00588385 |
| | response | | | | |
| GO:0042542 | response to hydrogen peroxide | 10 | 10/269 | 146/18723 | 0.00659515 |
| GO:0031960 | response to corticosteroid | 10 | 10/269 | 167/18723 | 0.01102469 |
| GO:0043281 | regulation of cysteine-type | 10 | 10/269 | 209/18723 | 0.03074569 |
| | endopeptidase activity involved in | | | | |
| | apoptotic process | | | | |
| GO:0001505 | regulation of neurotransmitter | 10 | 10/269 | 213/18723 | 0.0338307 |
| | levels | | | | |
| GO:1903305 | regulation of regulated secretory | 9 | 9/269 | 136/18723 | 0.01102469 |
| | pathway | | | | |
| GO:0050768 | negative regulation of neurogenesis | 9 | 9/269 | 140/18723 | 0.01220005 |
| GO:0032355 | response to estradiol | 9 | 9/269 | 141/18723 | 0.01241135 |
| GO:0051961 | negative regulation of nervous | 9 | 9/269 | 145/18723 | 0.0149786 |
| | system development | | | | |
| GO:0051384 | response to glucocorticoid | 9 | 9/269 | 148/18723 | 0.015648 |
| GO:1903900 | regulation of viral life cycle | 9 | 9/269 | 148/18723 | 0.015648 |
| GO:0050792 | regulation of viral process | 9 | 9/269 | 164/18723 | 0.02374296 |
| GO:0002221 | pattern recognition receptor | 9 | 9/269 | 172/18723 | 0.02995881 |
| | signaling pathway | | | | |
| GO:0010721 | negative regulation of cell | 9 | 9/269 | 180/18723 | 0.03683661 |
| | development | | | | |
| GO:0043123 | positive regulation of I-kappaB | 9 | 9/269 | 186/18723 | 0.04388257 |
| | kinase/NF-kappaB signaling | | | | |
| GO:0045071 | negative regulation of viral genome | 8 | 8/269 | 56/18723 | 0.00086822 |
| | replication | | | | |
| GO:0032722 | positive regulation of chemokine | 8 | 8/269 | 71/18723 | 0.0024544 |
| | production | | | | |
| GO:0045069 | regulation of viral genome | 8 | 8/269 | 85/18723 | 0.0046685 |
| | replication | | | | |
| GO:0048525 | negative regulation of viral process | 8 | 8/269 | 92/18723 | 0.00674818 |
| GO:0032642 | regulation of chemokine production | 8 | 8/269 | 98/18723 | 0.00806699 |
| GO:0032602 | chemokine production | 8 | 8/269 | 99/18723 | 0.00806699 |
| GO:0048661 | positive regulation of smooth | 8 | 8/269 | 104/18723 | 0.01011236 |
| | muscle cell proliferation | | | | |
| GO:0014812 | muscle cell migration | 8 | 8/269 | 110/18723 | 0.01212044 |
| GO:0019079 | viral genome replication | 8 | 8/269 | 131/18723 | 0.02374296 |
| GO:0010977 | negative regulation of neuron | 8 | 8/269 | 137/18723 | 0.02929644 |
| | projection development | | | | |
| GO:0030879 | mammary gland development | 8 | 8/269 | 137/18723 | 0.02929644 |
| GO:0050671 | positive regulation of lymphocyte | 8 | 8/269 | 137/18723 | 0.02929644 |
| | proliferation | | | | |
| GO:0032946 | positive regulation of mononuclear | 8 | 8/269 | 138/18723 | 0.02995881 |
| | cell proliferation | | | | |
| GO:0001890 | placenta development | 8 | 8/269 | 144/18723 | 0.03597724 |
| GO:0070665 | positive regulation of leukocyte | 8 | 8/269 | 150/18723 | 0.043775 |
| | proliferation | | | | |
| GO:0002753 | cytoplasmic pattern recognition | 7 | 7/269 | 60/18723 | 0.00399649 |
| | receptor signaling pathway | | | | |
| GO:0014910 | regulation of smooth muscle cell | 7 | 7/269 | 89/18723 | 0.015648 |
| | migration | | | | |
| GO:0014909 | smooth muscle cell migration | 7 | 7/269 | 97/18723 | 0.02144666 |
| GO:0007173 | epidermal growth factor receptor | 7 | 7/269 | 108/18723 | 0.03096394 |
| | signaling pathway | | | | |
| GO:0022612 | gland morphogenesis | 7 | 7/269 | 118/18723 | 0.04569764 |
| GO:0001704 | formation of primary germ layer | 7 | 7/269 | 121/18723 | 0.0502262 |
| GO:0038127 | ERBB signaling pathway | 7 | 7/269 | 121/18723 | 0.0502262 |
| GO:0018149 | peptide cross-linking | 6 | 6/269 | 35/18723 | 0.00268973 |
| GO:0032728 | positive regulation of interferon- | 6 | 6/269 | 39/18723 | 0.00336289 |
| | beta production | | | | |
| GO:0032608 | interferon-beta production | 6 | 6/269 | 56/18723 | 0.01090625 |
| GO:0032648 | regulation of interferon-beta | 6 | 6/269 | 56/18723 | 0.01090625 |
| | production | | | | |
| GO:0032481 | positive regulation of type I | 6 | 6/269 | 58/18723 | 0.01174232 |
| | interferon production | | | | |
| GO:0021675 | nerve development | 6 | 6/269 | 77/18723 | 0.02938488 |
| GO:0046928 | regulation of neurotransmitter | 6 | 6/269 | 86/18723 | 0.043775 |
| | secretion | | | | |
| GO:0039528 | cytoplasmic pattern recognition | 5 | 5/269 | 34/18723 | 0.00942454 |
| | receptor signaling pathway in | | | | |
| | response to virus | | | | |
| GO:0090050 | positive regulation of cell migration | 5 | 5/269 | 35/18723 | 0.01037325 |
| | involved in sprouting angiogenesis | | | | |
| GO:0048483 | autonomic nervous system | 5 | 5/269 | 47/18723 | 0.02285789 |
| | development | | | | |
| GO:0021545 | cranial nerve development | 5 | 5/269 | 52/18723 | 0.02995881 |
| GO:0001706 | endoderm formation | 5 | 5/269 | 54/18723 | 0.0338307 |
| GO:0030199 | collagen fibril organization | 5 | 5/269 | 61/18723 | 0.0502262 |
| GO:1902287 | semaphorin-plexin signaling | 4 | 4/269 | 12/18723 | 0.00336289 |
| | pathway involved in axon guidance | | | | |
| GO:1902285 | semaphorin-plexin signaling | 4 | 4/269 | 13/18723 | 0.00434529 |
| | pathway involved in neuron | | | | |
| | projection guidance | | | | |
| GO:0140374 | antiviral innate immune response | 4 | 4/269 | 15/18723 | 0.00659515 |
| GO:0021783 | preganglionic parasympathetic fiber | 4 | 4/269 | 16/18723 | 0.00701583 |
| | development | | | | |
| GO:0048486 | parasympathetic nervous system | 4 | 4/269 | 18/18723 | 0.00917146 |
| | development | | | | |
| GO:0072567 | chemokine (C-X-C motif) ligand 2 | 4 | 4/269 | 20/18723 | 0.01129431 |
| | production | | | | |
| GO:2000341 | regulation of chemokine (C-X-C | 4 | 4/269 | 20/18723 | 0.01129431 |
| | motif) ligand 2 production | | | | |
| GO:1900017 | positive regulation of cytokine | 4 | 4/269 | 26/18723 | 0.02111551 |
| | production involved in | | | | |
| | inflammatory response | | | | |
| GO:0039529 | RIG-I signaling pathway | 4 | 4/269 | 27/18723 | 0.0230333 |
| GO:0021602 | cranial nerve morphogenesis | 4 | 4/269 | 29/18723 | 0.02768835 |
| GO:0018401 | peptidyl-proline hydroxylation to 4- | 3 | 3/269 | 10/18723 | 0.01653479 |
| | hydroxy-L-proline | | | | |
| GO:0048251 | elastic fiber assembly | 3 | 3/269 | 10/18723 | 0.01653479 |
| GO:0021561 | facial nerve development | 3 | 3/269 | 11/18723 | 0.01957287 |
| GO:0021604 | cranial nerve structural organization | 3 | 3/269 | 11/18723 | 0.01957287 |
| GO:0021610 | facial nerve morphogenesis | 3 | 3/269 | 11/18723 | 0.01957287 |
| GO:0042167 | heme catabolic process | 3 | 3/269 | 11/18723 | 0.01957287 |
| GO:0046149 | pigment catabolic process | 3 | 3/269 | 11/18723 | 0.01957287 |
| GO:2000343 | positive regulation of chemokine | 3 | 3/269 | 11/18723 | 0.01957287 |
| | (C-X-C motif) ligand 2 production | | | | |
| GO:0006787 | porphyrin-containing compound | 3 | 3/269 | 12/18723 | 0.02311841 |
| | catabolic process | | | | |
| GO:0021559 | trigeminal nerve development | 3 | 3/269 | 12/18723 | 0.02311841 |
| GO:0033015 | tetrapyrrole catabolic process | 3 | 3/269 | 12/18723 | 0.02311841 |
| GO:0018158 | protein oxidation | 3 | 3/269 | 14/18723 | 0.03096394 |
| GO:0015671 | oxygen transport | 3 | 3/269 | 15/18723 | 0.03620671 |
| GO:0019511 | peptidyl-proline hydroxylation | 3 | 3/269 | 15/18723 | 0.03620671 |
| GO:0048532 | anatomical structure arrangement | 3 | 3/269 | 17/18723 | 0.04896731 |

DMBT1 is a large protein (260 kD) with thirteen tandem repeated scavenger receptor cysteine-rich domains. To examine if DMBT1 might recognize the M13 phage and the down-regulation of DMBT1 could lead to enhanced phage transduction, the conditioned medium from the parental HeLa cells or the DMBT1-knockout HeLa cells was coated on the plate, and the M13 phage binding was evaluated by ELISA. The results were shown in FIG. 3J. It was found that the phage binding was significantly weaker to the DMBT1-knockout conditioned medium, suggesting the role of DMBT1 in recognizing the M13 phage. Due to the phage sequestered by DMBT1, the phage transduction efficiency was about 5% higher in the DMBT1-knockout HeLa as compared to the parental HeLa (FIGS. 3K and 3L).

### Example 4: Phage Transduction Efficiency was Inversely Correlated with the Phage Length

In this example, the transduction efficiency was improved by engineering the phage particles. For example, it was investigated if the size of the phage might influence multiple processes of phage transduction, including internalization, intracellular trafficking, escape of viral defense, and dsDNA conversion.

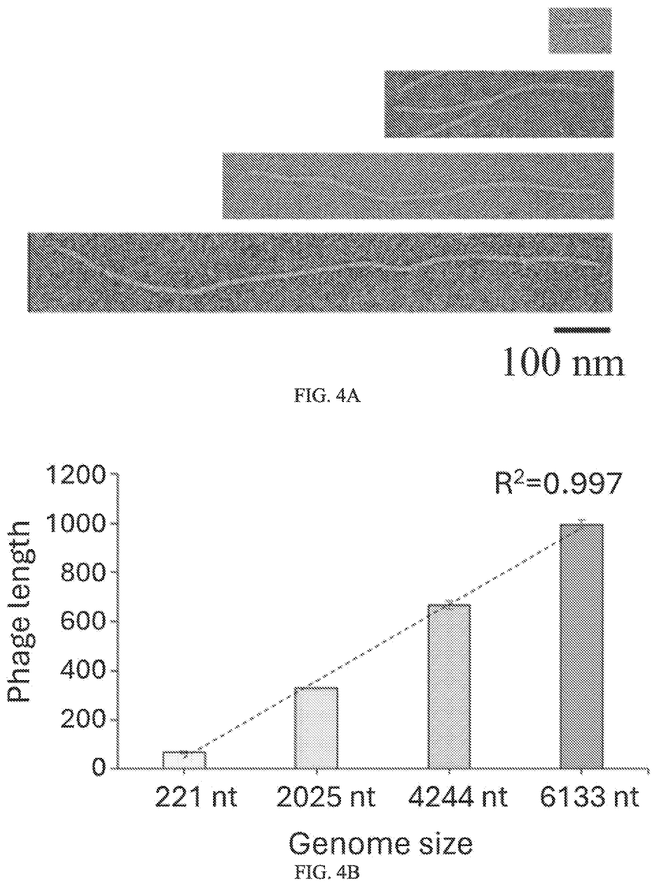

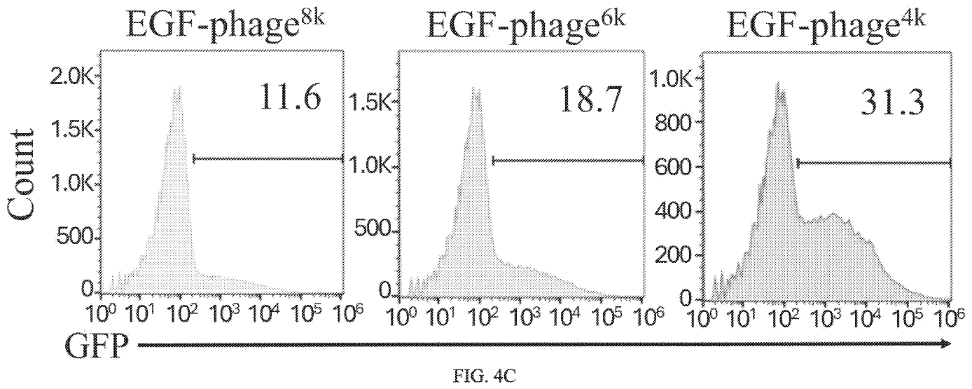

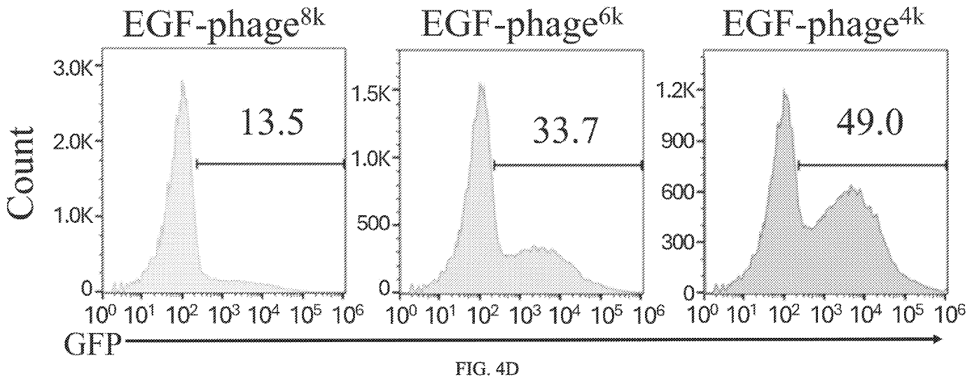

The EGF-phage^{6k} (GFP) as mentioned in Example 1 carried a phagemid with 6,133 nt, which contained a GFP reporter, an ampicillin resistance marker, a neomycin resistance marker, an f1 origin, and a ColE1 plasmid origin. On this basis, two other phages, EGF-phage^{4k} (GFP) (4,244 nt) and EGF-phage^{8k} (GFP) (8,361 nt), were created by deleting the neomycin resistance marker and inserting a stuffer sequence, respectively. The electron-microscope images showed that the length of the phage was linearly proportional to the size of the phagemid (FIGS. 4A and 4B). It was found that the transduction efficiency was inversely correlated with the phage size. The EGF-phage^{4k} (GFP) achieved 31.3% GFP+cells, significantly outperforming the EGF-phage^{6k}(GFP) (18.7%) and the EGF-phage^{8k} (GFP) (11.6%) (FIG. 4C).

The phage used in this example was generated by a standard helper (M13KO7) with a defective f1 origin, which is known to contribute about 10% helper phage in the total phage production. To completely avoid the helper interference, an f1-less helper plasmid was constructed by removing the defective f1 origin. Further, as shown in FIG. 4D, the phage generated by the f1-less helper greatly improved the transduction efficiency for all three lengths of phage with the shortest phage EGF-phage^{4k} (GFP) up to 49.0%.

### Example 5: Design of TransPhage with Minimal Length and Optimum Transduction Efficiency by Origin Engineering

To further minimize the phage size, the GFP cassette (CMV promoter+GFP+poly-A tail) was inserted between the initiator and the packaging signal (FIG. 5A) to create the EGF-phage^{2k}(GFP) (1,960 nt) with the length around 300 nm, which was less than the half size of EGF-phage^{4k}(GFP) (4,244 nt). FIG. 5B showed that the transduction efficiency of EGF-phage^{2k} (GFP) was similar to EGF-phage^{4k} (GFP) when an equal production unit of phage (total phage from 3 mL bacteria culture) was applied.

Comparing the phage origins between EGF-phage^{2k} and EGF-phage^{4k} (FIG. 5A), it was found that two replication elements, namely domain B and loop B-C, were absence in the EGF-phage^{2k}. The insertion of domain B (EGF-phage^{2k+donainB}) led to an improved transduction when the phage was generated by the M13KO7 helper, which carried the domain A but not the domain B in its defective f1 origin (FIG. 5C, left panel). These results suggested that EGF-phage^{2k+domainB} produced more favorably than EGF-phage^{2k} when the helper competition was present. However, when the phage was generated by the f1-less helper, the transduction efficiency of EGF-phage^{2k} and EGF-phage^{2k+donainB} became equally good (FIG. 5C, right panel).

On top of the domain B, an additional incorporation of the loop B-C (EGF-phage^{2k+domainB+loopBC}) resulted in a better transduction efficiency (74.8%), which outperformed EGF-phage^{2k+domainB} (62.1%) even when the f1-less helper was used for the phage production (FIG. 5D). The EGF-phage^{2k+domains+loopBC} was named as “TransPhage” herein, which carried the domain A, the domain B, the gene of interest, the packaging signal, and the loop B-C in the described order (FIG. 5E; SEQ ID NO. 1).

Furthermore, the transduction efficiency of EGF-TransPhage (GFP) (2,156 nt) was compared to other three phages with different sizes, EGF-phage^{4k} (GFP) (4,244 nt), EGF-phage^{6k}(GFP) (6,133 nt), and EGF-phage^{8k} (GFP) (8,361 nt). When an equal multiplicity of infection (MOI=10⁷) was applied to the PrimPol-HeLa^{hi} cells, an inverse correlation between the phage size and the transduction efficiency was observed (FIG. 5F). Also, by the experiments using an equal weight of phage (FIG. 5G) with the unit that was relevant to the clinical applications, it was found that the superiority of TransPhage over other phages was even more profound because it had the greater encapsulating efficiency for the gene of interest (weight^{GOI}/weight^{phage}). Besides the PrimPol-HeLa^{hi} cells, the EGF-TransPhage carrying mCherry or membrane-bound human Fc also showed great transduction efficiency to the parental HeLa cells, ranging from 40% to 67% depending on the gene delivered (FIG. 5H).

### Example 6: TransPhage Showed Comparable Transduction Efficiency and Superior Specificity to the AAV Vectors

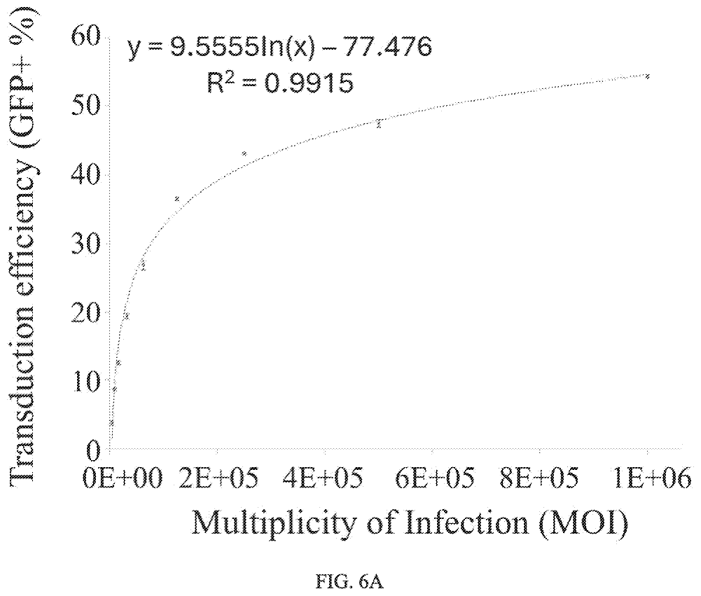

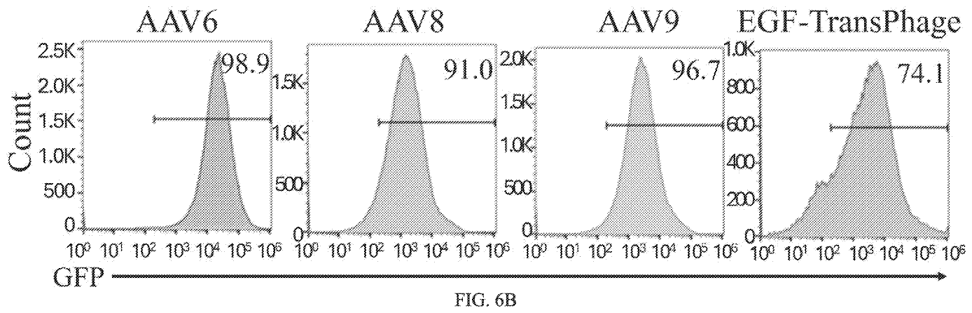

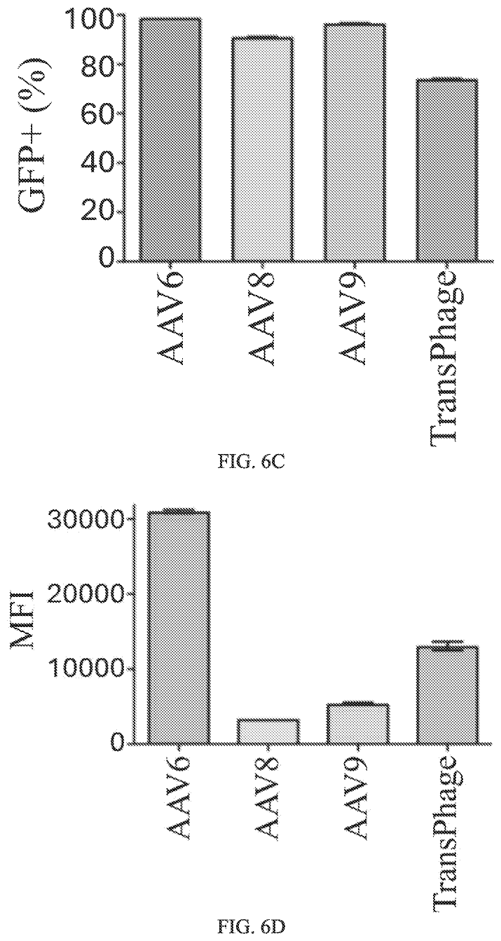

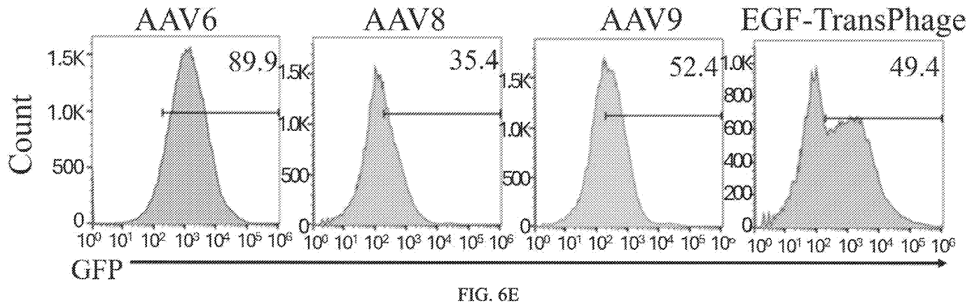

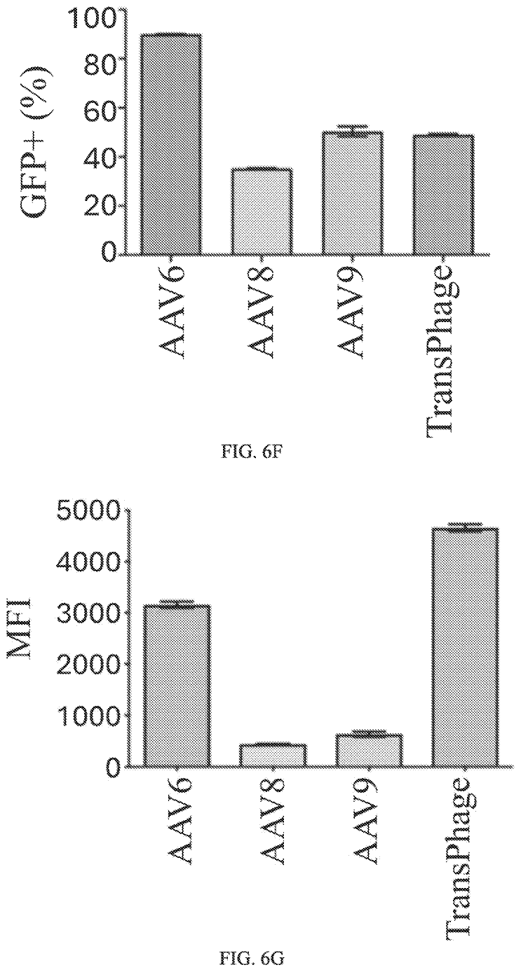

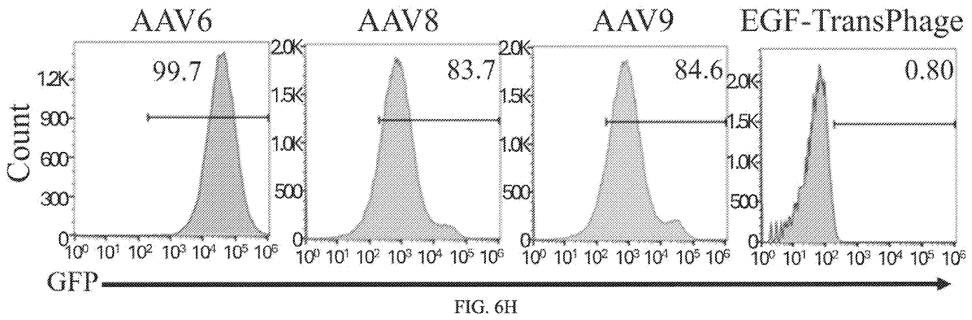

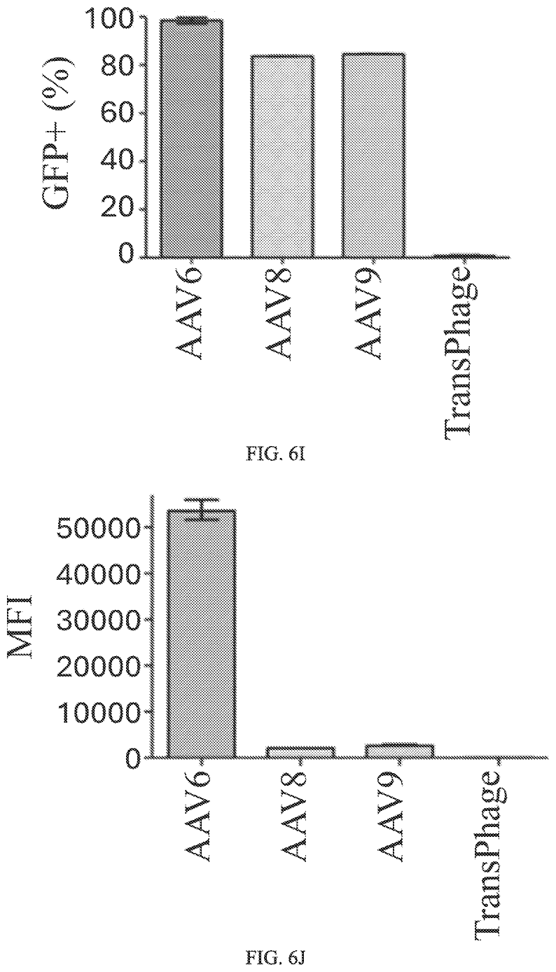

In this example, the comparison of the transduction efficiency of TransPhage with that of the AAV vectors was performed. Three serotypes of AAVs (AAV6, AAV8, and AAV9) and EGF-TransPhage, all of which carried the GFP reporter driven by the CMV promoter, were applied to the PrimPol-HeLa^{hi} cells at the MOI of 103 to 10⁷. The phage transduction efficiency showed a logarithmic relationship to the MOI (FIG. 6A).

At the MOI of 10⁷, all three AAVs showed GFP+cells >90%, whereas EGF-TransPhage reached about 75% (FIGS. 6B and 6C). Nevertheless, the mean fluorescence intensity (MFI) of EGF-TransPhage was only behind AAV6 but surpassed AAV8 and AAV9 (FIG. 6D). It was also noticed that AAV6, but not AAV8, AAV9, or EGF-TransPhage, was toxic to the cells and significantly slowed down the cell growth. At the MOI of 10⁶, AAV6 remained a high GFP+population (about 90%), whereas AAV8, AAV9, and EGF-TransPhage exhibited 30% to 50% (FIGS. 6E and 6F). Nevertheless, EGF-TransPhage showed highest MFI, followed by AAV6, AAV9, and AAV8 (FIG. 6G). Interestingly, it was also found that the EGF-TransPhage transduction tended to result in a binary GFP population (GFP+ and GFP-peaks, as shown in FIG. 6E), whereas the AAV transductions led to more homogeneous GFP distributions.

Ideally, a successful gene therapy requires not only a high transduction efficiency to the disease cells, but also a low/negligible transduction efficiency to the healthy cells. A serious drawback of AAV vectors is their promiscuous infectivity toward many human cell types. On the other hand, TransPhage is customized to selectively infect the cells that overexpress the target antigen.

In this regard, HEK293T was used as a surrogate of the healthy cells, which did not express the target antigen EGFR. Indeed, all AAV vectors showed very high transduction efficiencies to HEK293T (>80%, as shown in FIGS. 6H to 6J), which were comparable to their infectivity in HeLa. On the contrary, EGF-TransPhage exhibited non-detectable transduction in HEK293T. Overall, these results demonstrated that EGF-TransPhage had comparable transduction efficiency and superior specificity compared to the AAV vectors.

### Example 7: TransPhage Successfully Delivered Therapeutic Genes into Cancer Cells with High Efficiency and Efficacy

For the cancer therapy, several therapeutic genes were implemented in EGF-TransPhage, and their anti-tumor efficacy in vitro was tested.

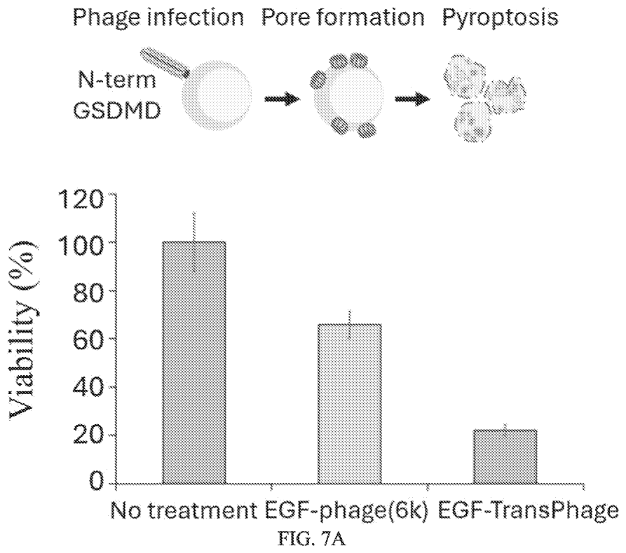

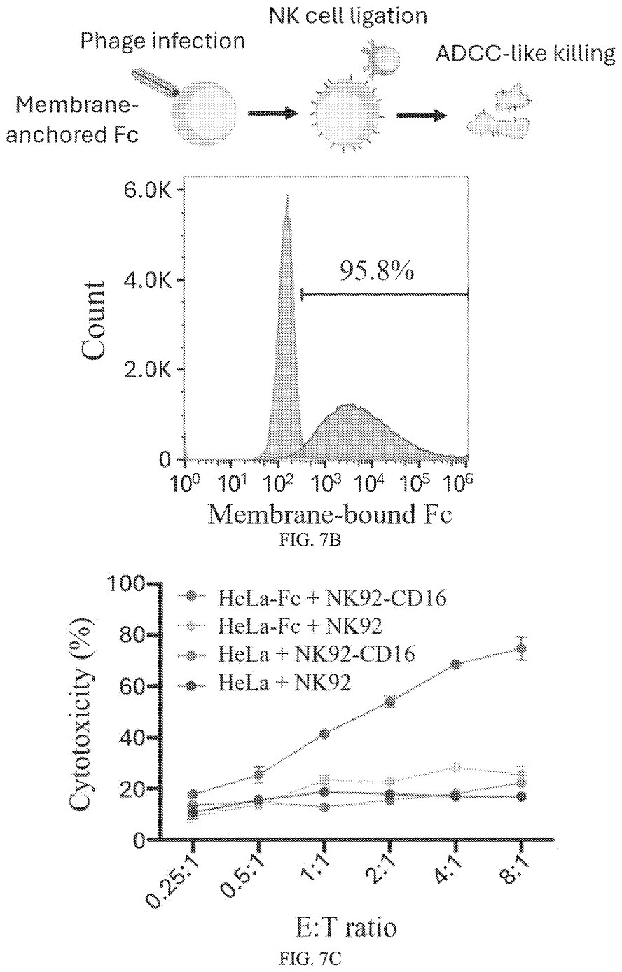

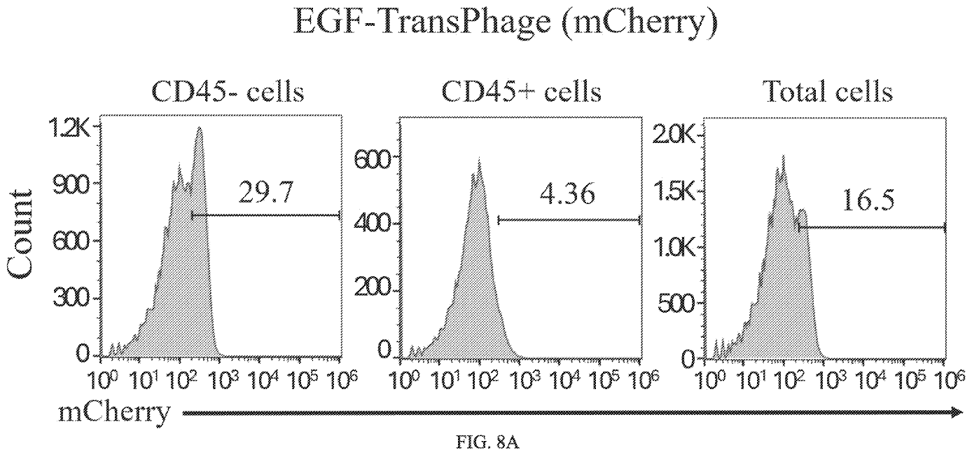

GSDMD is a human protein that induces the pyroptosis when the pore-forming domain at the N-terminus is cleaved from the auto-inhibition domain at the C-terminus by caspases^{[12]}. EGF-TransPhage (2,264 nt) and EGF-phage^{6k} (6,241 nt) carrying the N-terminal GSDMD gene were created, respectively, and subjected to induce pyroptosis in the PrimPol-HeLa^{hi} cells. FIG. 7A showed that EGF-TransPhage (GSDMD) successfully eliminated most cells, whereas EGF-phage^{6k} (GSDMD) exhibited detectability but less toxicity.

On the other hand, for the immunogen therapy, a membrane-bound fragment crystallizable region (Fc) was genetically introduced into cancer cells. The membrane-bound Fc might induce the antibody-dependent cell-mediated cytotoxicity (ADCC)-like killing by natural killer cells (NK cells) or the antibody-dependent cellular phagocytosis (ADCP)-like killing by phagocytes.

The membrane-bound-Fc gene was implemented into EGF-TransPhage, resulting in a 2,384-nt ssDNA encapsulated. Notably, a very high transduction efficiency was achieved in the PrimPol-HeLa^{hi} cells (95.8%, as shown in FIG. 7B) as well as the parental HeLa cells (67.1%, as shown in FIG. 5H). In the killing experiments, a potent and dose-dependent cytotoxicity was observed when a CD16-expressing NK-92 cell line was co-cultured with the HeLa cells that expressed the membrane-bound Fc (FIG. 7C). Either the NK-92 cells without CD16 expression or the HeLa cells without the membrane-bound Fc expression completely abolished the cytotoxicity.

### Example 8: Immunogen Therapy Delivered by TransPhage Suppressed the Xenograft Tumors in Nude Mice

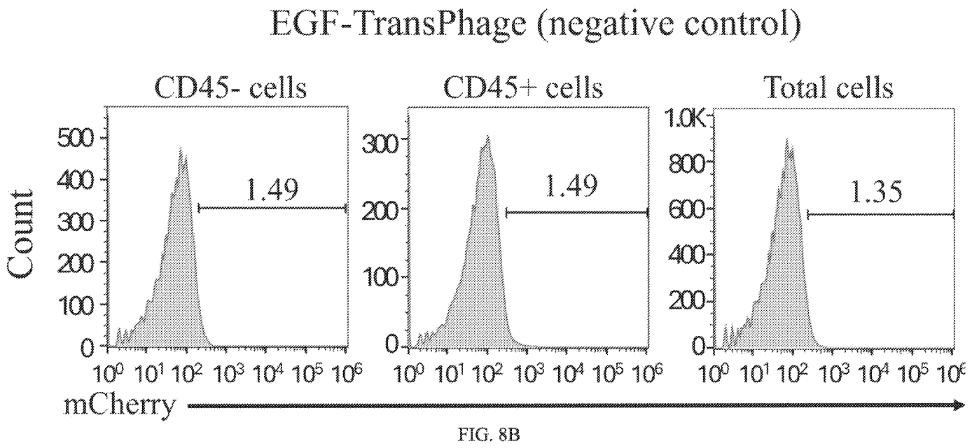

Given the remarkable efficacy of the NK-mediated immunogen therapy in vitro, the anti-tumor activity was further examined in the xenograft mouse model. In this example, the safety of TransPhage in mice was first checked, and no significant weight loss was observed after 4 injections of the TransPhage. Subsequently, the transduction efficiency in PC3 xenograft tumors was evaluated by intratumoral injection of EGF-TransPhage (mCherry). The transduction efficiency was preferentially higher in the CD45− cells (29.7%) than in the CD45+cells (4.36%), presumably due to the high EGFR expression in PC3 (FIGS. 8A and 8B).

Next, the human prostate cancer cell line PrimPol-PC3 was subcutaneously inoculated in nude mice, and four groups of treatments (EGF-TransPhage carrying mCherry, membrane-bound human Fc, membrane-bound mouse Fe, and no treatment) were delivered intratumorally every other day for four times when the tumor size was about 100 mm³.

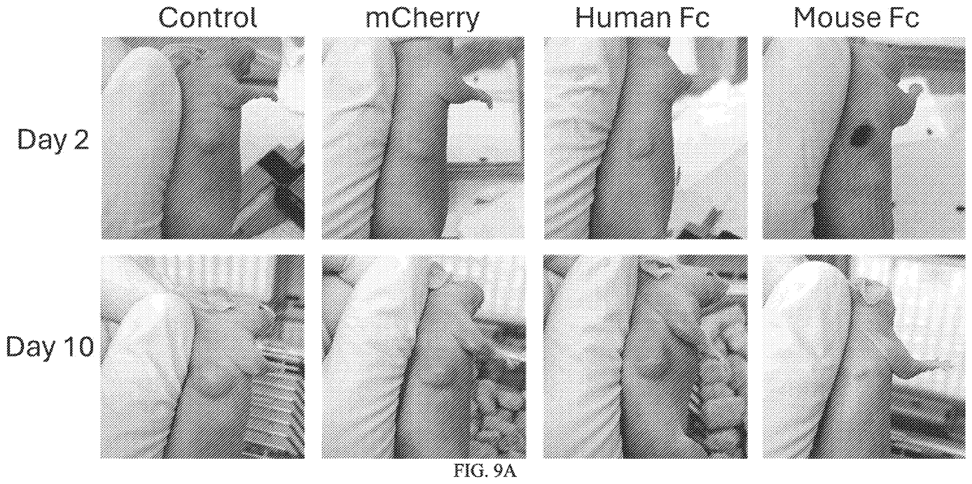

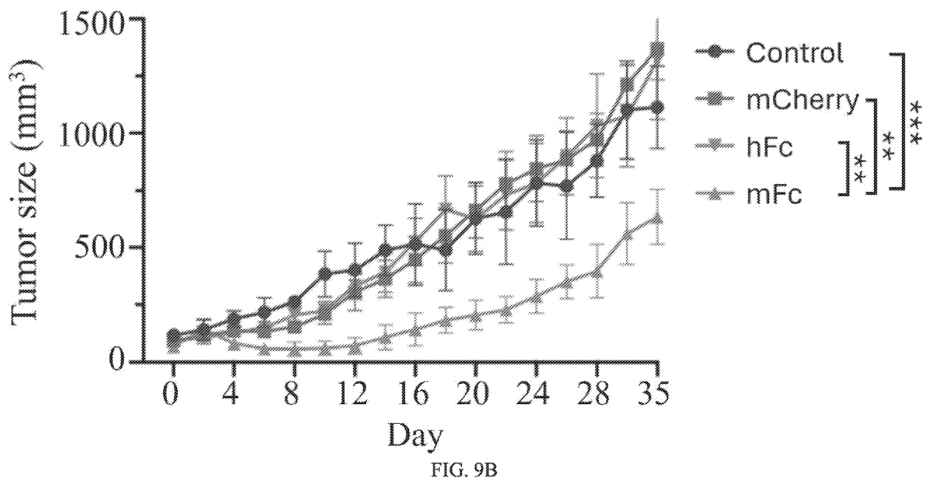

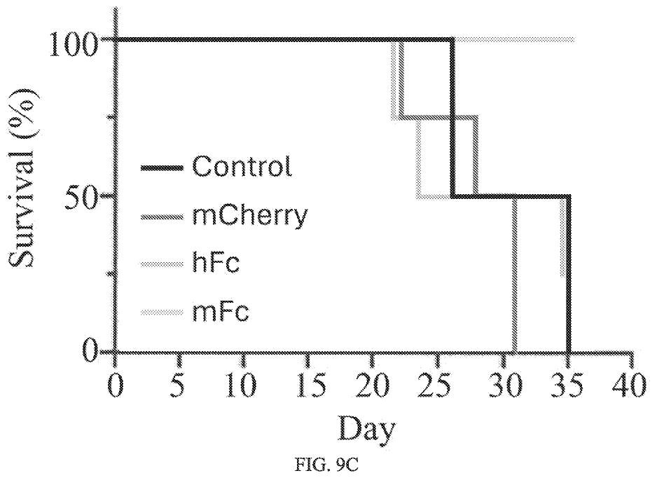

As soon as 2 days after the first administration, it was found that the appearance of tumors turned black only in the EGF-TransPhage (mFc) group, presumably due to the induction of necrosis (FIG. 9A). The black tissue eventually turned into a scab, which fell off naturally on day 10. At this point, the tumors in the EGF-TransPhage (mFc) group were almost non-palpable.

Also, the tumor growth curves showed that the EGF-TransPhage (mFc) treatment significantly inhibited the tumor progression and enhanced the survival rate, while the EGF-TransPhage (hFc) and the EGF-TransPhage (mCherry) treatments did not show any efficacy as compared to the control (FIGS. 9B and 9C). These results suggested that only the membrane-bound mouse Fc, but not the membrane-bound human Fc, induced the potent ADCC-like and/or ADCP-like responses in the mouse model. It was worth noting that the experiments were performed in the immunocompromised mice without lymphocytes. In an immunocompetent background, it was expected that the ADCC-like and ADCP-like killing would stimulate the T cells for further tumor clearance.

From the above, these results reveal that through shortening the phage length with the restructured f1 origin, the engineered bacteriophage particle may achieve 40% to 67% and 75% to 95% transduction efficiencies to the parental HeLa cells and the PrimPol-HeLa^{hi} cells, respectively. In contrast, the highest transduction efficiency of the M13 vectors of the prior act is only about 10%. Accordingly, this is a dramatic improvement achieved by the present disclosure that has made the gene delivery efficiency of bacteriophage comparable or even superior to the AAV vectors.

Further, unlike the human viral vectors that generally infect a broad spectrum of cells, the engineered bacteriophage particle of the present disclosure provides a highly specific tropism by targeting the surface antigen using displayed ligands or antibodies. This property is useful for clinical applications because most TAAs, such as HER2 or EGFR, are also present in healthy cells with moderate levels. Hence, the engineered bacteriophage particle of the present disclosure is effective in gene therapy among other human viral vectors.

It is obvious to a person skilled in the art that with the advancement of technology, the basic idea may be implemented in various ways. The embodiments are thus not limited to the examples described above; instead, they may vary within the scope of the claims.

The embodiments described hereinbefore may be used in any combination with each other. Several of the embodiments may be combined together to form a further embodiment. A method disclosed herein may comprise at least one of the embodiments described hereinbefore. It will be understood that the benefits and advantages described above may relate to one embodiment or may relate to several embodiments. The embodiments are not limited to those that solve any or all of the stated problems or those that have any or all of the stated benefits and advantages.

### REFERENCE

[1] Ma, C. C., Wang, Z. L., Xu, T., He, Z. Y., and Wei, Y. Q. (2020). The approved gene therapy drugs worldwide: from 1998 to 2019. Biotechnol. Adv. 40, 107502.
[2] Russell, S., Bennett, J., Wellman, J A., Chung, D. C., Yu, Z. F., Tillman, A., Wittes, J., Pappas, J., Elci, O., McCague, S., et al. (2017). Efficacy and safety of voretigene neparvovec (AAV2-hRPE65v2) in patients with RPE65-mediated inherited retinal dystrophy: a randomised, controlled, open-label, phase 3 trial. Lancet 390, 849-860.
[3] Day, J. W., Finkel, R. S., Chiriboga, C. A., Connolly, A. M., Crawford, T. O., Darras, B. T., Iannaccone, ST., Kuntz, N. L., Pena, L. D. M., Shieh, P. B., et al. (2021). Onasemnogene abeparvovec gene therapy for symptomatic infantile-onset spinal muscular atrophy in patients with two copies of SMN2 (STRIVE): an open-label, single-arm, multicentre, phase 3 trial. Lancet Neurol. 20, 284-293.
[4] Lisowski, L., Dane, A. P., Chu, K., Zhang, Y., Cunningham, S. C., Wilson, E. M., Nygaard, S., Grompe, M., Alexander, I. E., and Kay, M. A. (2014). Selection and evaluation of clinically relevant AAV variants in a xenograft liver model. Nature 506, 382-386.
[5] Chan, K. Y., Jang, M. J., Yoo, B. B., Greenbaum, A., Ravi, N., Wu, W. L., Sanchez-Guardado, L., Lois, C., Mazmanian, S. K., Deverman, B E., et al. (2017). Engineered AAVs for efficient noninvasive gene delivery to the central and peripheral nervous systems. Nat. Neurosci. 20, 1172-1179.
[6] Bowles, D. E., McPhee, S. W. J., Li, C. W., Gray, S. J., Samulski, J. J., Camp, A. S., Li, J., Wang, B., Monahan, P. E., Rabinowitz, J. E., et al. (2012). Phase 1 gene therapy for Duchenne muscular dystrophy using a translational optimized AAV vector. Molecular Therapy 20, 443-455.
[7] Larocca, D., Jensen-Pergakes, K., Burg, M. A., and Baird, A. (2001). Receptor-targeted gene delivery using multivalent phagemid particles. Molecular Therapy 3, 476-484.
[8] Chen, C. C., Sun, C. P., Ma, H. I., Fang, C. C., Wu, P. Y., Xiao, X., and Tao, M. H. (2009). Comparative study of anti-hepatitis B virus RNA interference by double-stranded adeno-associated virus serotypes 7, 8, and 9. Mol. Ther. 17, 352-359.
[9] Burg, M. A., Jensen-Pergakes, K., Gonzalez, A. M., Ravey, P., Baird, A., and Larocca, D. (2002). Enhanced phagemid particle gene transfer in camptothecin-treated carcinoma cells. Cancer Research 62, 977-981.
[10] Ebner, R. and Derynck, R. (1991). Epidermal growth factor and transforming growth factor-alpha: differential intracellular routing and processing of ligand-receptor complexes. Cell Regul. 2, 599-612.
[11] Garcia-Gomez, S., Reyes, A., Martinez-Jimenez, MI., Chocron, E. S., Mouron, S., Terrados, G., Powell, C., Salido, E., Mendez, J., Holt, I. J., et al. (2013). PrimPol, an archaic primase/polymerase operating in human cells. Mol. Cell 52, 541-553.
[12] Ding, J., Wang, K., Liu, W., She, Y., Sun, Q., Shi, J., Sun, H., Wang, D. C., and Shae, F. (2016). Pore-forming activity and structural autoinhibition of the gasdermin family. Nature 535, 111-116.

1. An engineered phagemid for gene delivery, comprising:
an f1 origin of replication including a phage packaging signal and domain A; and
a transgene expression cassette configured to express a transgene in a eukaryotic cell,
wherein the engineered phagemid has a length less than about 8,000 nucleotides and is free from encoding an M13 phage structural protein required for M13 phage assembly.

2. The engineered phagemid of claim 1, wherein the transgene expression cassette is integrated into the f1 origin of replication.

3. The engineered phagemid of claim 2, wherein the f1 origin of replication comprises a first nucleotide sequence having at least 70% sequence identity to SEQ ID NO. 10 and a second nucleotide sequence having at least 70% sequence identity to SEQ ID NO. 11, and wherein the first nucleotide sequence and the second nucleotide sequence serve as signal for initiation of replication of the engineered phagemid and have the same function as SEQ ID NOs. 10 and 11, respectively.

4. The engineered phagemid of claim 2, wherein the f1 origin of replication comprises at least one nucleotide sequence having at least 70% to one of SEQ ID NOs. 2 to 6, and wherein the at least one nucleotide sequence serves as signal for initiation of replication of the engineered phagemid and has the same function as the corresponding nucleotide sequence of SEQ ID NOs. 2 to 6.

5. The engineered phagemid of claim 2, wherein the f1 origin of replication is genetically modified to further include a truncated domain A.

6. The engineered phagemid of claim 5, wherein the f1 origin of replication further includes domain B and loop B-C, and the domain A, the domain B, the transgene expression cassette, the phage packaging signal, the loop B-C, and the truncated domain A are comprised in order of 5′ to 3′ in the f1 origin of replication.

7. The engineered phagemid of claim 5, wherein the f1 origin of replication further includes domain B, and the domain A, the domain B, the transgene expression cassette, the phage packaging signal, and the truncated domain A are comprised in order of 5′ to 3′ in the f1 origin of replication.

8. The engineered phagemid of claim 5, wherein the f1 origin of replication is genetically modified to lack at least one of loop B-C and domain B.

9. The engineered phagemid of claim 8, wherein the f1 origin of replication is genetically modified to lack loop B-C and domain B, and the domain A, the transgene expression cassette, the phage packaging signal, and the truncated domain A are comprised in order of 5′ to 3′ in the f1 origin of replication.

10. The engineered phagemid of claim 1, which has the length ranging from about 1,000 nt to about 6,500 nt.

11. The engineered phagemid of claim 1, wherein the transgene expression cassette comprises a functional element required for expression of the transgene in the eukaryotic cell, and the functional element is at least one of a promoter and a poly-A sequence.

12. The engineered phagemid of claim 11, wherein the promoter is a mammalian expression promoter.

13. The engineered phagemid of claim 11, wherein the promoter is selected from the group consisting of a cytomegalovirus major immediate-early promoter, a simian virus 40 promoter, a β-actin promoter, an albumin promoter, an elongation factor 1-α promoter, a PγK promoter, an MFG promoter, and a Rous sarcoma virus promoter.

14. A vector system of producing an engineered bacteriophage particle for gene delivery, the vector system comprising:
the engineered phagemid of claim 1; and
a helper plasmid comprising an M13 phage genome, a prokaryotic origin of replication, and a nucleic acid encoding a cell-targeting ligand,
wherein the helper plasmid encodes an M13 phage structural protein required for packaging the engineered phagemid in a prokaryotic cell, thereby forming the engineered bacteriophage particle for gene delivery.

15. The vector system of claim 14, wherein the M13 phage genome comprises genes of pI, pII, pIII, pIV, pV, pVI, pVII, pVIII, pIX, and pX.

16. The vector system of claim 15, wherein the nucleic acid encoding the cell-targeting ligand is fused into the pIII gene.

17. The vector system of claim 14, wherein the helper plasmid comprises a disrupted f1 origin of replication or is free of an f1 origin of replication.

18. An engineered bacteriophage particle for gene delivery, comprising:
the engineered phagemid of claim 1;
an M13 phage capsid encapsulating the engineered phagemid of claim 1; and
a cell-targeting ligand displayed on the M13 phage capsid.

19. The engineered bacteriophage particle of claim 18, which has a length ranging from about 300 nm to about 600 nm.

20. The engineered bacteriophage particle of claim 18, wherein the cell-targeting ligand is displayed on a pIII protein of the M13 phage capsid.

21. The engineered bacteriophage particle of claim 18, wherein the cell-targeting ligand is selected from the group consisting of a peptide, an antibody, an antibody fragment, and a non-proteinaceous molecule.

22. The engineered bacteriophage particle of claim 18, wherein the cell-targeting ligand is a tumor-targeting ligand.

23. A method of delivering a transgene to a eukaryotic cell, comprising contacting the eukaryotic cell with the engineered bacteriophage particle of claim 18.

24. The method of claim 23, wherein the eukaryotic cell is a PrimPol-overexpressing and/or DMBT1 knockout cell.

25. A method of preventing or treating a disease susceptible to amelioration by gene therapy, comprising administering a therapeutically effective amount of the engineered bacteriophage particle of claim 18 to a subject in need thereof.

26. The method of claim of 25, wherein the disease susceptible to amelioration by gene therapy is a cancer or a genetic disorder.

27. The method of claim of 26, wherein the cancer is selected from the group consisting of skin cancer, breast cancer, head and neck cancer, lung cancer, stomach cancer, pancreatic cancer, ovarian cancer, cervical cancer, uterine cancer, kidney cancer, bladder cancer, colon cancer, prostate cancer, central nervous system cancer, ocular melanoma, neuroblastoma, multiple myeloma, and lymphoma.

28. The method of claim of 26, wherein the genetic disorder is selected from the group consisting of Duchenne muscular dystrophy, limb-girdle muscular dystrophy, spinal muscular atrophy, myotubular myopathy, Pompe disease, glycogen storage disease, Leber's congenital amaurosis, amyotrophic lateral sclerosis, color blindness, type 1 diabetes, and Fabry disease.


SEQUENCE LISTING (11 sequences)

SEQ ID NO: 1 - DNA, 519 bp, synthetic construct
  source 1..519: mol_type = other DNA
ccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaagcttgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaa

SEQ ID NO: 2 - DNA, 94 bp, synthetic construct
  source 1..94: mol_type = other DNA
ccatcgccctgatagacggtttttcgccctttgacgttggagtccacgttctttaatagtggactcttgttccaaactggaacaacactcaacc

SEQ ID NO: 3 - DNA, 96 bp, synthetic construct
  source 1..96: mol_type = other DNA
ctatctcggtctattcttttgatttataagggattttgccgatttcggcctattggttaaaaaatgagctgatttaacaaaaatttaacgcgaatt

SEQ ID NO: 4 - DNA, 99 bp, synthetic construct
  source 1..99: mol_type = other DNA
aagcttgtacgcgccctgtagcggcgcattaagcgcggcgggtgtggtggttacgcgcagcgtgaccgctacacttgccagcgccctagcgcccgctcc

SEQ ID NO: 5 - DNA, 101 bp, synthetic construct
  source 1..101: mol_type = other DNA
ccgtcaagctctaaatcgggggctccctttagggttccgatttagtgctttacggcacctcgaccccaaaaaacttgattagggtgatggttcacgtagtg

SEQ ID NO: 6 - DNA, 84 bp, synthetic construct
  source 1..84: mol_type = other DNA
ggccatcgccctgatagacggtttttcgccctttgacgttggagtccacgttctttaatagtggactcttgttccaaactggaa

SEQ ID NO: 7 - protein, 9 aa, synthetic construct
RRRHIVRKR

SEQ ID NO: 8 - protein, 9 aa, synthetic construct
AAAHIVAAA

SEQ ID NO: 9 - DNA, 21 bp, synthetic construct
  source 1..21: mol_type = other DNA
gcgagtggaggtcctataccg

SEQ ID NO: 10 - DNA, 190 bp, synthetic construct
  source 1..190: mol_type = other DNA
ccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatt

SEQ ID NO: 11 - DNA, 329 bp, synthetic construct
  source 1..329: mol_type = other DNA
aagcttgtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaa